(19) United States
(12) Patent Application Publication
(10) Pub. No.: US 2011/0111007 A1
(43) Pub. Date: May 12, 2011

(54) INHIBITION OF RETINAL CELL DEGENERATION OR NEOVASCULARIZATION BY CERIUM OXIDE NANOPARTICLES

(76) Inventors: James F. McGinnis, Edmond, OK (US); Xiaohong Zhou, Arlington, MA (US); Lily L. Wong, Oklahoma City, OK (US); Sudipta Seal, Orlando, FL (US)

(21) Appl. No.: 12/772,523

(22) Filed: May 3, 2010

Related U.S. Application Data
(63) Continuation-in-part of application No. 11/412,665, filed on Apr. 27, 2006, now Pat. No. 7,727,559.
(60) Provisional application No. 60/716,630, filed on Sep. 13, 2005.
(60) Provisional application No. 60/676,043, filed on Apr. 29, 2005.

(51) Int. Cl. A61F 2/14; A61K 33/24; A61P 27/02; A61P 27/06; A61P 29/00; A61P 19/02; A61P 1/00; A61P 17/00; A61K 9/14; B82Y 5/00

(52) U.S. Cl. 424/427; 424/617; 424/489; 977/915

(57) ABSTRACT

The presently claimed and disclosed inventive concept(s) provides methods for reducing, reversing or inhibiting retinal cell degeneration, or neovascularization in tissues of a mammalian subject having a pathological condition involving neovascularization, by administration in vivo of nanoceria particles (cerium oxide nanoparticles) to the subject. The method of the presently claimed and disclosed inventive concept(s) is useful, for example, for reducing, treating, reversing or inhibiting degeneration of retinal cells such as photoreceptor cells or neovascularization in ocular tissue such as the retina, macula or cornea; or other tissues such as, but not limited to, skin, synovial tissue, intestinal tissue, or bone. In addition, the method of the presently claimed and disclosed inventive concept(s) is useful for reducing or inhibiting neovascularization in a neoplasm (tumors), which can be benign or malignant and, where malignant, can be a metastatic neoplasm. As such, the presently claimed and disclosed inventive concept(s) is directed to using compositions containing nanoceria particles to reduce, treat, reverse or inhibit angiogenesis in a mammalian subject.

### CROSS-REFERENCE TO RELATED APPLICATIONS

The present application is a continuation-in-part of U.S. Ser. No. 12/429,650, filed Apr. 24, 2009, which claims the benefit under 35 U.S.C. §119(e) of U.S. Provisional Application Ser. No. 61/125,602, filed Apr. 25, 2008.

The present application also claims the benefit under 35 U.S.C. §119(e) of U.S. Provisional Application Ser. No. 61/174,678, filed May 1, 2009.

The present application is also a continuation-in-part of U.S. Ser. No. 11/142,665, filed Apr. 27, 2006, which claims the benefit under 35 U.S.C. §119(e) of U.S. Provisional Application Ser. No. 60/716,630, filed Sep. 13, 2005, and U.S. Provisional Application Ser. No. 60/676,043, filed Apr. 29, 2005.

The entireties of each of the applications listed herein are hereby expressly incorporated herein by reference.

### STATEMENT REGARDING FEDERALLY SPONSORED RESEARCH OR DEVELOPMENT

Not applicable.

### BACKGROUND

Angiogenesis (also referred to herein as neovascularization) is the process whereby new blood vessels are formed. Angiogenesis occurs normally during embryogenesis and development, and occurs in fully developed organisms during wound healing and placental development. In addition, angiogenesis occurs in various pathological conditions, including in ocular diseases such as diabetic retinopathy and macular degeneration due to neovascularization, in conditions associated with tissue inflammation such as rheumatoid arthritis and inflammatory bowel disease, and in cancer, where blood vessel formation in the growing tumor provides oxygen and nutrients to the tumor cells, as well as providing a route via which tumor cells metastasize throughout the body. Since millions of people around the world are afflicted by these diseases, a considerable effort has been made to understand the mechanisms involved in angiogenesis in the hope that such an understanding will allow the development of methods for detecting and inhibiting such undesirable angiogenesis.

Angiogenesis occurs in response to stimulation by one or more known growth factors, and also may involve other as yet unidentified factors. Endothelial cells, which are the cells that line mature blood vessels, normally do not proliferate. However, in response to an appropriate stimulus, the endothelial cells become activated and begin to proliferate and migrate into unvascularized tissue forming new blood vessels. In some cases, precursor cells can be activated to differentiate into endothelial cells which form new blood vessels.

Blood vessels are surrounded by an extracellular matrix. In addition to stimulation by growth factors, neovascularization depends on interaction of the endothelial cells with the extracellular matrix, as well as with each other. The activation of endothelial cells by growth factors and the migration into and interaction with the extracellular matrix and with each other is dependent on cell surface receptors expressed by the endothelial cells. These cell surface receptors, which include growth factor receptors and integrins, interact specifically with particular molecules.

In pathological conditions such as age-related macular degeneration and diabetic retinopathy, decreasing availability of oxygen to the retina results in a hypoxic condition that stimulates the secretion of angiogenic growth factors such as vascular endothelial growth factors (VEGF), which induce abnormal migration and proliferation of endothelial cells within tissues of the eye. Such neovascularization in ocular tissues can induce corneal scarring, retinal detachment, fluid accumulation in the subretinal space, and macular edema, each of which can adversely affect vision and lead to blindness.

Angiogenesis also is associated with the progression and exacerbation of inflammatory diseases, including psoriasis, rheumatoid arthritis, osteoarthritis, and inflammatory bowel diseases such as ulcerative colitis and Crohn's disease. In inflammatory arthritic disease, for example, influx of lymphocytes into the region surrounding the joints stimulates angiogenesis in the synovial lining. The increased vasculature provides a means for greater influx of leukocytes, which facilitate the destruction of cartilage and bone in the joint. Neovascularization that occurs in inflammatory bowel disease results in similar effects in the bowel.

The growth of capillaries into atherosclerotic plaques in the coronary arteries represents another pathological condition associated with growth factor induced angiogenesis. Excessive blood flow into neovascularized plaques can result in rupture and hemorrhage of the blood-filled plaques, releasing blood clots that can result in coronary thrombosis.

The involvement of angiogenesis in such diverse diseases as cancer, ocular disease and inflammatory diseases has led to an effort to identify methods for specifically inhibiting angiogenesis as a means to treat these diseases. For cancer patients, such methods of treatment can provide a substantial advantage over currently used methods such as chemotherapy, which kill or impair not only the target tumor cells, but also normal cells in the patient, particularly proliferating normal cells such as blood cells, epithelial cells, and cells lining the intestinal lumen. Such non-specific killing by chemotherapeutic agents results in side effects that are, at best, unpleasant, and can often result in unacceptable patient morbidity, or mortality. In fact, the undesirable side effects associated with cancer therapies often limit the treatment a patient can receive.

For other pathological conditions associated with abnormal angiogenesis such as diabetic retinopathy, there are no effective treatments. However, even if retinal transplantation is performed, the new retina would be subject to the same conditions that resulted in the original retinopathy. Thus, there exists a need for novel methods of inhibiting and treating neovascularization in patients suffering from pathological conditions characterized by this condition. The presently claimed and disclosed inventive concept(s) satisfies this need and provides related advantages as well in the treatment of other disease conditions identified herein.

The retina is the part of the eye that is sensitive to light. The macula lutea is the region of the retina that allows us to read and recognize faces. Diseases of the macula, such as age-related macular degeneration (AMD) and diabetic macular edema, account for a major proportion of legal blindness. To combat these diseases, a variety of accepted and experimental medications are employed via systemic routes or local, invasive surgical procedures.

Diabetic retinopathy (DR), a leading cause of blindness in type 1 and type 2 diabetics, is a complication of diabetes which produces damage to the blood vessels inside the retina. Diabetic retinopathy can have four stages: (1) mild nonproliferative retinopathy, wherein microaneurysms in the retina's blood vessels occur; (2) moderate nonproliferative retinopathy, wherein some blood vessels feeding the retina become blocked; (3) severe nonproliferative retinopathy, wherein many blood vessels to the retina are blocked, depriving several areas of the retina with their blood supply; and (4) proliferative retinopathy, wherein new, abnormal, thin-walled and fragile-walled blood vessels grow to supply blood to the retina, but which new blood vessels may leak fluids including but not limited to blood to produce severe vision loss and blindness. Hemorrhages can occur more than once, often during sleep. Fluid can also leak into the center of the macula at any stage of diabetic retinopathy and cause macular edema and blurred vision. Almost all Americans who have had diabetes for longer than ten years will develop some stage of diabetic retinopathy, and about half of the people with proliferative retinopathy also have macular edema.

Macular degeneration is a degeneration of the macular region of the retina in the eye. Degeneration of the macula causes a decrease in acute vision and can lead to eventual loss of acute vision. The wet form of macular degeneration is related to abnormal growth of blood vessels in the retina that can leak blood and can cause damage to photoreceptor cells. Age-related macular degeneration is a collection of clinically recognizable ocular findings that can lead to blindness. Macular degeneration is a group of diseases. There are two basic types of macular degeneration, including “wet” and “dry”. In wet macular degeneration, there is an abnormal growth of new blood vessels (neovascularization). These new blood vessels break and leak fluid, causing damage to the central retina. This form of macular degeneration is often associated with aging. Approximately 85% of macular degeneration cases are dry macular degeneration. Vision loss can result from the accumulation of deposits in the retina called druzen, and from the death of photoreceptor cells in the retina. This process can lead to thinning and drying of the retina.

The findings of AMD include the presence of druzen, retinal pigment epithelial disturbance, including pigment clumping and/or dropout, retinal pigment epithelial detachment, geographic atrophy, subretinal neovascularization and disciform scar. Age-related macular degeneration is a leading cause of presently incurable blindness, particularly in persons over 55 years of age. Approximately one in four persons age 65 or over have signs of age-related maculopathy, and about 35% of persons age 75 or over have some form of advanced macular degeneration with vision loss.

Druzen are ophthalmoscopically visible, yellow-white hyaline excrescences or nodules of Bruch's membrane. Bruch's membrane lies beneath the retina and the adjacent retina pigment epithelium layer. Fat accumulates in Bruch's membrane with age and may contribute to the formation of druzen. Druzen can occur in two forms. One form comprises hard, small (less than about 60 micrometers in diameter) objects which do not increase with age and which do not predispose to macular degeneration. Another form comprises soft, large (more than about 63 micrometers in diameter) objects which enlarge and become confluent with age. The soft, large druzen may predispose to macular degeneration, and are commonly seen in eyes of people with advanced macular degeneration in at least their other eye.

Druzen may be metabolic waste products from various layers of the retina such as from the retina, retina pigment epithelium, and choriocapillaris. Druzen may be yellow, white, gray, refractile, and/or pink. Druzen may be small, medium or large in size. Druzen may be regular or irregular, or symmetrical or asymmetrical in shape. A patient who has druzen and who suffers complications in one eye may suffer no complications in the other eye. Complications may comprise one or more conditions selected from the group consisting of retina pigment epithelium atrophy, choroid neovascularization, retina detachment serous, and retina detachment hemorrhagic. Druzen may affect contrast sensitivity, and may reduce the eye's ability to see sufficiently to allow a person to read in dim light or to see sufficient detail to permit a person to drive an automobile safely at night.

A contributing and indicating factor of advanced macular degeneration is neovascularization of the choroid tissue underlying the photoreceptor cells in the macula. Healthy mature ocular vasculature is normally quiescent and exists in a state of homeostasis in which a balance is maintained between positive and negative mediators of angiogenesis in development of new vasculature. Macular degeneration, particularly in its advanced stages, is characterized by the pathological growth of new blood vessels in the choroid underlying the macula. Angiogenic blood vessels in the subretinal choroid can leak vision obscuring fluids, leading to blindness.

The major causes of blindness in the United States are glaucoma, AMD, cataracts, DR, and retinitis pigmentosa (RP) which translates into more than 38 million citizens having some form of an age related eye disease. In developed countries, cataracts are routinely surgically removed and vision is restored with the insertion of an artificial lens. There are no cures for most forms of the other blinding diseases, the severity of which increases with age and dramatically decreases the quality of life for these patients. Even for patients with an inherited blindness, such as RP, vision worsens with age. Nearly 1 out of 3 individuals over the age of 75 will develop some form of AMD and with the aging of the “Baby Boomers” generation, this means a dramatic increase in patient numbers. Nearly 200,000 individuals in the USA develop AMD each year. About 2 million Americans over the age of 40 have significant vision loss due to AMD while an additional 8 million have a high risk of vision loss. DR has some symptoms very similar to wet AMD including neovascular growth in the eye and subfoveal macular edema. About 4 million Americans age 40 and older have DR and >80% of patients who have diabetes for more than ten years will develop DR. Further, because Native American Indians develop diabetes at a much higher rate than the general population, DR is becoming a progressively increasing problem in states such as Oklahoma which have large numbers of Native Americans. The annual economic cost to the USA for adult vision loss is major at about $50 billion per year.

As noted above, AMD is classified roughly into two categories based on the absence or presence of choroidal neovascularizations which grow into the eye. Most patients have the dry form (85%) whereas about 15% have the wet form. The dry form is characterized by the accumulation of debris (druzen) between the retinal pigment epithelia and Bruch's membrane which effectively eliminates the benefits of the choroidal blood supply to the adjacent photoreceptor cells. The dry form can lead to the wet form, but optionally may not, and patients may still retain some retinal function throughout life. In the wet form, the presence of sub-macular neovascular vessels leads to retinal edema, ruptured blood vessels, the death of the cones in the macula and eventual blindness. Current therapeutic treatments for wet AMD involve intravitreal injections of monoclonal antibodies against Vascular Endothelial Growth Factor (VEGF) every 6-8 weeks. There are no treatments which have proven successful for dry AMD. Recently, a subform of AMD was recognized, called Retinal Angiomatous Proliferation (RAP), in which neovascular lesions occurred within the photoreceptor cell layer as a result of neovessels growing from the retinal vasculature through the photoreceptors, the RPE and Bruch's membrane where they joined choroidal neovascular tufts. These patients represent about 15% of the vascular form of AMD. It is an object of the presently claimed and disclosed inventive concept(s) to develop a therapeutic treatment for blinding diseases, such as RAP.

Mammalian cells produce cellular energy in mitochondria by using oxygen to metabolize molecular substrates. The vast majority of the products of this oxidative metabolism are beneficial while about 3% are highly toxic compounds such as singlet oxygen, the hydroxide ion, and hydrogen peroxide. These Reactive Oxygen Species (ROS) can react with and damage almost any type of molecule within the cell including proteins, DNA, RNA and lipids. Another major source of intracellular ROS is NADPH oxidase which activates the STAT3 pathway which upregulates retinal VEGF. The normal antioxidant cellular defenses against ROS include catalytic proteins such as superoxide dismutase, heme oxygenase and thioredoxin as well as small molecules like glutathione, and NADPH. Oxidative stress occurs when the level of ROS exceeds the ability of the cells' antioxidant defenses to scavenge or destroy them. Because of the close proximity of the intra-mitochondrial components to the ROS, it is not surprising that they bear the brunt of damage from ROS and with decreased oxidative phosphorylation they produce less energy but more ROS.

As indicated herein, there are many diseases which result in the programmed cell death of retinal photoreceptor cells thus leading to blindness. These include illnesses which are known to be inherited such as retinitis pigmentosa as well as many which have a genetic component but which may be environmentally induced or are of questionable origin such as diabetic retinopathy and AMD. Interestingly, irrespective of the primary cause, all of these diseases are thought to share some common nodes, including oxidative stress caused by a chronic or acute rise in ROS and apoptosis. The retina has the highest rate of oxygen metabolism, is constantly bombarded with photons of light, and is therefore exposed to a higher concentration of ROS than any other tissue of the body. Neurodegeneration within the retina is not unlike neurodegeneration within other areas of the central nervous system. Even in the albino rat model of light-induced degeneration of photoreceptor cells, initiation of apoptosis proceeds through the intracellular production of ROS. Strong evidence that oxidative damage is a primary cause of AMD was recently presented by Hollyfield et al. (“Oxidative damage-induced inflammation initiates age-related macular degeneration’, Nat. Med. 2008; 14:194-198).

Treatments for diseases involving neovascularization and retinal degeneration are highly desirable and it is to these treatments that the presently claimed and disclosed inventive concept(s) is directed.

### SUMMARY OF THE DISCLOSURE

The presently claimed and disclosed inventive concept(s) provides methods for reducing, reversing or inhibiting retinal cell degeneration, or neovascularization in tissues of a mammalian subject having a pathological condition involving neovascularization, by administration in vivo of nanoceria particles (cerium oxide nanoparticles) to the subject. The method of the presently claimed and disclosed inventive concept(s) is useful, for example, for reducing, treating, reversing or inhibiting degeneration of retinal cells such as rod and cone photoreceptor cells or neovascularization in ocular tissue such as the retina, macula or cornea; or other tissues such as, but not limited to, skin, synovial tissue, intestinal tissue, or bone. In addition, the method of the presently claimed and disclosed inventive concept(s) is useful for reducing or inhibiting neovascularization in a neoplasm (tumors), which can be benign or malignant and, where malignant, can be a metastatic neoplasm. As such, the presently claimed and disclosed inventive concept(s) is directed to using compositions containing nanoceria particles to reduce, treat, reverse or inhibit angiogenesis in a mammalian subject.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows photomicrographs of results from four assays for ROS and ROS-induced changes in VLDLr Knock Out (VLDLr KO) mouse retinas.

FIG. 2 includes Western Blots (A,C) and Densitometry graphs (B,D) showing that nanoceria (cerium oxide nanoparticles, a.k.a., CNP) inhibit the rise in VEGF in retinas of VLDLr KO mice. Actin was stained as a load control.

FIG. 3 presents immunofluorescence confocal photomicrographs which show localization of VEGF in retinas and effects of nanoceria thereon.

FIG. 4 shows micrographs of VLDLr KO retinal vasculature which show the presence of new blood vessels (”bleb” at arrowheads). These blebs are reduced by nanoceria treatment.

FIG. 5 shows cut and flattened eyecups of normal (C57) and VLDLr KO mice, with and without treatment with nanoceria. The bright dots in the VLDLr eyecup(B) are choroidal neovascular tufts which are greatly reduced in the CNP treated eye(C).

FIG. 6 shows micrographs of VLDLr KO vascular defects in saline and CNP (CeO₂) injected eyes.

FIG. 7 is a graph quantitating the effects of treatment with nanoceria on numbers of choroidal tufts and the intraretinal vascular blebs at postnatal days 14, 21 and 28 for CNP treated and untreated eyes.

FIG. 8 is a graph showing that nanoceria treatment causes regression of retinal vascular lesions which were present prior to treatment.

FIG. 9 is a graph showing that nanoceria downregulate VEGF within one week in mature VLDLr KO retinas.

FIG. 10 (A1-E1, A2-E2, A3-E3) are photomicrographs showing that the subretinal and intraretinal lesions in the VLDLr−/−mice arise developmentally the same as those in patients with a form of AMD called retinal angiomatous proliferation (RAP).

FIG. 11 (A-H) are micrographs, graphs, and blots showing regression of various existing illicit developmental features in VLDLr−/−mice.

FIG. 12 is a graph showing CNP inhibit inherited retinal degeneration in the tubby retina. Quantitative histology shows that injection of CNP at P7 inhibits degeneration of the tubby outer nuclear layer (ONL) when measured at P34 (34 days post-natal).

FIG. 13 show that CNP improve retinal function by more than two fold over saline controls. Electroretinography (ERG) data showing the scotopic ERG data for (a) the A-wave and (B) the B-wave of C57BL/6J (wild type control) and saline injected and saline+CNP injected tubby mice. The controls are off scale (box shows value) in order to readily see saline and CNP treated mice. All mice were given intraperitoneal injections of 20 μl of saline, or 1 mM CNP in saline, every day from day P7 (7 days post-natal) through P27 (27 days post natal). Systemic injections are effective.

FIG. 14 shows results of Electroretinography of normal, heterozygous and homozygous tubby retinas injected intracardially with 30 μl of 1 nM CNP on postnatal days 10, 20 and 30 (P10, P20, P30) and assayed on day 34 (P34). The data indicates that there is a significant protection of retinal function for both the A-wave and B-wave ERGs in the CNP injected tubby mice (n=6) compared with saline-treated tub/tub (n=6) mice at P34.

FIG. 15 contains micrographs of retinal cross-sections of tubby mice treated with CNP which decrease the number of retinal cells undergoing programmed cell death as revealed by the TUNEL assay for apoptotic cells.

FIG. 16 are graphs showing decreases in the activities of Caspase-9 and Caspase-3 in retinas of tubby mice intracardially injected with nanoceria.

FIG. 17 is a graph showing Caspase-8 activity in retinas of saline-or CNP-treated tub/tub mice and heterozygotes. These data indicate that the CNP are almost as effective as the presence of a normal tubby gene.

FIG. 18 shows cross-sectional micrographs of and ethidine assay demonstrating decreased oxidative stress in CNP treated tubby retinas and saline treated tubby and normal wild type retinas injected intracardially or intravitreally.

FIG. 19 shows cross-sectional micrographs of the dichlorofluorescein assay for reactive oxygen species in retinas of tubby and wild-type mice treated with saline or nanoceria injected via IV or IC.

### DETAILED DESCRIPTION

While the presently claimed and disclosed inventive concept(s) is now described below in connection with certain embodiments in the following description and examples so that aspects thereof may be more fully understood and appreciated, it is not intended to limit the presently claimed and disclosed inventive concept(s) to these particular embodiments and examples. On the contrary, it is intended to cover all alternatives, modifications and equivalents as may be included within the scope of the presently claimed and disclosed inventive concept(s) as defined herein. Thus, the following examples and embodiments will serve to illustrate the practice of this presently claimed and disclosed inventive concept(s), it being understood that the particulars shown are by way of example and for purposes of illustrative discussion of various embodiments of the presently claimed and disclosed inventive concept(s) only and are presented in the cause of providing what is believed to be the most useful and readily understood description of formulation procedures as well as of the principles and conceptual aspects of the presently claimed and disclosed inventive concept(s).

The presently claimed and disclosed inventive concept(s) provides methods for reducing, treating, reversing, or inhibiting retinal cell death and/or neovascularization in a tissue of a mammalian subject having a pathological condition involving retinal cell degeneration and/or neovascularization by in vivo administration of nanoceria particles (cerium oxide nanoparticles, a.k.a., CNPs) in the subject.

The method of the presently claimed and disclosed inventive concept(s) is useful, for example, for reducing, treating, reversing or inhibiting neovascularization in pathological conditions associated with ocular tissue such as the retina, macula or cornea, or in other tissues including but not limited to, skin, synovial tissue, intestinal tissue, or bone. In addition, the method of the presently claimed and disclosed inventive concept(s) is useful for reducing, reversing or inhibiting neovascularization in neoplasms (tumors), which can be benign or malignant and, where malignant, can be metastatic neoplasms. As such, the presently claimed and disclosed inventive concept(s) provides compositions, which contain nanoceria particles and are useful for reducing, reversing, or inhibiting angiogenesis associated with such pathological condition in a mammalian subject.

The methods of the presently claimed and disclosed inventive concept(s) are also useful in reducing or inhibiting degeneration of retinal cells in a subject, and particularly of retinal rod and cone photoreceptor cells in mammalian retinas by in vivo administration of nanoceria particles in the subject.

In particular, the presently claimed and disclosed inventive concept(s) demonstrates effectiveness of CNPs applied via systemic administration to reduce the risk of ocular damage (such as retinal detachment, ocular infection) that may associate with intravitreal injections. The potential ease of administration enables the treatment to be carried out by general health practitioners instead of ophthalmologists. The cost of therapy is substantially lower.

The pathological conditions treated by the method of the presently claimed and disclosed inventive concept(s) include, but are not limited to, those of: the eye, such as diabetic retinopathy or macular degeneration; the skin, such as a hemangioma or psoriasis; a joint, such as rheumatoid arthritis or osteoarthritis; or the intestine, such as Crohn's disease or ulcerative colitis; or can be a tumor, which can be benign or malignant.

The nanoceria of the presently claimed and disclosed inventive concept(s) which are useful for reducing or inhibiting retinal cell degeneration or a pharmaceutical composition thereof containing the nanoceria can be used for treating any pathological condition that is characterized, at least in part, by retinal cell degeneration or by angiogenesis. The nanoceria can be administered by various systemic routes including, for example, parenterally, including intravenously, intramuscularly, subcutaneously, intraorbitally, intracapsularly, intrasynovially, intraperitoneally, intracisternally or by passive or facilitated absorption through the skin using, for example, a skin patch or transdermal iontophoresis. Furthermore, the composition can be administered by injection, intubation, via a suppository, orally or topically, the latter of which can be passive, for example, by direct application of an ointment or powder containing the composition, or active, for example, using a nasal spray or inhalant. The pharmaceutical composition also can be incorporated, if desired, into liposomes, microspheres or other polymer matrices as discussed elsewhere herein.

The presently claimed and disclosed inventive concept(s) further provides methods of reducing, treating, reversing or inhibiting neovascularization (angiogenesis) in a tissue in an individual, by administering to the individual the nanoceria particles of the presently claimed and disclosed inventive concept(s), thereby reducing or inhibiting angiogenesis in the tissue in the individual and, consequently, reducing the severity of the pathological condition exhibiting the angiogenesis. The condition can be any pathological condition associated with angiogenesis, including a neoplasm, which can be a malignant neoplasm, for example, a carcinoma such as breast carcinoma, colon carcinoma, ovarian carcinoma or pancreatic carcinoma, or a sarcoma, mesothelioma, teratocarcinoma, an astrocytoma, glioblastoma, or other neoplasm, including a metastatic malignant neoplasm. The agent can be administered by various routes, for example, intravenously or directly into the region to be treated, for example, directly into a neoplastic tumor; via eye drops or intravitreal injection, where the pathological condition involves the eye; or intrasynovially, where the condition involves a joint, or via other methods as discussed elsewhere herein.

Without wishing to be bound by theory, it is believed that in the presently claimed and disclosed inventive concept(s), the reduction, reversal, or inhibition of neovascularization in pathological conditions of ocular tissue such as retina, macula or cornea; of skin such as occurs with psoriasis; of synovial tissue; of bone; or of intestinal tissue; or of benign or malignant neoplasms occurs by reducing the reactive oxygen species (ROS) in the tissue.

The term “pathological condition” is used broadly herein to mean any abnormal physical or physiological condition characterized, at least in part, by neovascularization. Such pathological conditions include inflammation, neoplasms, ocular diseases such as diabetic retinopathy and macular degeneration associated with neovascularization, skin diseases such as psoriasis and hemangiomas, gingivitis, arthritic conditions such as rheumatoid arthritis and osteoarthritis, and inflammatory bowel diseases.

The term “neoplasm” is used broadly herein to mean any new, pathological tissue growth. For purposes of the presently claimed and disclosed inventive concept(s), a neoplasm generally results in the formation of a tumor, which is characterized, in part, by angiogenesis. A neoplasm can be benign, for example, a hemangioma, glioma, teratoma, and the like, or can be malignant, for example, a carcinoma, sarcoma, glioblastoma, astrocytoma, neuroblastoma, retinoblastoma, and the like. The term “tumor” is used generally to refer to a benign or malignant neoplasm, and the term “cancer” is used generally to refer to a malignant neoplasm, which may or may not be metastatic. Malignant neoplasms that can be treated using a method of the presently claimed and disclosed inventive concept(s) include, for example, carcinomas such as lung cancer, breast cancer, prostate cancer, cervical cancer, pancreatic cancer, colon cancer and ovarian cancer; and sarcomas such as osteosarcoma and Kaposi's sarcoma, provided the neoplasm is characterized, at least in part, by angiogenesis.

An individual to be treated using a method of the presently claimed and disclosed inventive concept(s) can be any individual exhibiting a neovascularization associated with a pathological condition or subject to retinal degeneration and, therefore, can be, for example, a vertebrate such as a mammal, including a human or other primate, dog, cat, horse, cow, sheep, or goat or any other mammal, particularly a commercially important animal or a domesticated animal and any other animal subject to diseases similar to those described herein. Treatment using the methods of the presently claimed and disclosed inventive concept(s) is considered to be successful when adverse clinical signs or symptoms associated in the subject with the pathological condition being treated are reduced, reversed, inhibited, or otherwise ameliorated. A reduction in the severity of a pathologic condition can be detected by various methods, including routine clinical tests such as blood tests, which can used to determine relevant enzyme levels or circulating antigen or antibody; imaging tests, which can be used to detect a decrease in the growth rate or size of a neoplasm; or an ophthalmic procedure, which can be used to identify a reduction in the number of blood vessels in the retina of a diabetic patient. Such clinical tests are selected based on the particular pathological condition being treated. A reduction in the severity of a pathological condition also can be detected based on comments made by the patient being treated, for example, that a patient suffering from arthritis feels less pain or has greater joint mobility, or that a patient with diabetic retinopathy or with macular degeneration due to neovascularization can see more clearly, or the like.

The nanoceria particle composition (cerium oxide nanoparticles) used in the presently claimed and disclosed inventive concept(s) generally will be in the form of a pharmaceutical composition comprising the nanoceria particles and a pharmaceutically acceptable carrier. Pharmaceutically acceptable carriers are well known in the art and include aqueous solutions such as physiologically buffered saline or other buffers or solvents or vehicles such as glycols, glycerol, oils such as olive oil or injectable organic esters. Pharmaceutically acceptable carriers also include inhalents, such as but not limited to, powders or liquids.

The total amount of the nanoceria composition can be administered to a subject as a single dose, over a relatively short period of time, or can be administered using a treatment protocol in which multiple doses are administered over a more prolonged period of time. The concentration and quantity of the nanoceria required in the treatment protocol depends on many factors including the age and general health of the subject as well as the route of administration, the number of treatments to be administered.

In one embodiment the presently claimed and disclosed inventive concept(s) comprises therapeutic treatments which eliminate the leaky neovessels, choroidal neovascularization and vascular lesions within the retinas of mammals with RAP and other ocular diseases characterized by neovascularization. The present therapeutic regimes also slow the progression of photoreceptor cell death, for example in all forms of AMD and DR as explained elsewhere herein in further detail. These treatments will dramatically improve the quality of life for millions of Americans and correspondingly reduce the $50 billion annual economic cost to the USA.

The presently claimed and disclosed inventive concept(s) in particular relates to methods of treatment of macular degeneration associated with subretinal neovascularization and a proliferation of neovascular tissue in the eye of a mammalian subject and/or with retinal rod and cone photoreceptor cell death and to methods of inhibiting or substantially reducing the rate of subretinal neovascularization and proliferation of neovascular tissue in the eye or retinal rod and cone photoreceptor cell death such as is associated with macular degeneration.

The presently claimed and disclosed inventive concept(s) also relates to methods of treatment of diabetic neuropathy, especially diabetic retinopathy associated with neovascularization, and to methods of inhibiting or substantially reducing the rate of proliferation of neovascular tissue in the eye associated with diabetic neuropathy.

Examples of cerium oxide nanoparticles that may be used in the presently claimed and disclosed inventive concept(s) include, but are not limited to, those described in U.S. Pat. Nos. 7,347,987 and 7,504,356 (each of which are expressly incorporated by reference herein) and include, but are not limited to, cerium oxides having the formulas CeO₂ and Ce₂O₃.

The nanoceria particles of the composition are administered in one embodiment at concentrations of 1 nM to 1000 μM, or from 1 nM to 100 μM, or from 1 nM to 10 μM, or from 1 nM to 1 μM, or from 1 nM to 100 nM, or from 1 nM to 50 nM, or from 1 nM to 10 nM, or from 10 nM to 10 μM, or from 100 nM to 10 μM. More particularly, the cerium oxide particles of the presently claimed and disclosed inventive concept(s) are further characterized as ultra-fine and are preferably in a size range of from approximately 1 nanometer in diameter to approximately 10 nanometers in diameter; more preferably from approximately 1 nm to approximately 7 nm.

Examples of various methods of administering therapeutic compositions to the eye, and which may be used to administer the nanoceria of the presently claimed and disclosed inventive concept(s), are described, for example, in U.S. Pat. No. 7,442,686, the entirety of which is hereby expressly incorporated herein by reference.

For example, the nanoceria particles may be administered by direct injection into the eye, intravenous, intraperitoneal, intramuscular, oral or topically on the eye or skin. The nanoceria will reduce damage to the eye caused by ocular angiogenesis and by retinal cell death in pathological conditions which include, but are not limited to, glaucoma, diabetic retinopathy, inherited retinal degeneration (for example, RP), AMD, retinal detachment or any disease or event which involves production of ROS, neovascularization, for example, including retinopathy of prematurity (ROP), neovascular glaucoma, macular edema, Sickle Cell retinopathy, choroidal neovascularization, retinal vascular diseases, and ocular oncology. These particles will preserve and prolong vision when administered in vivo.

Alternatively, administration of the nanoceria compositions of this presently claimed and disclosed inventive concept(s) is preferably by injection, such as by injection into an eye, preferably into a blood vessel that supplies blood to the eye or by microinjection into the macula by first penetrating the sclera, by topical application such as to a tissue of the eye such as the cornea or sclera, or by implantation such as by controlled release from a depot or implant comprising a pharmaceutically acceptable matrix or pharmaceutically acceptable carrier, which depot or implant is located proximal to the tissue of the eye, preferably proximal to or embedded into tissue comprising the posterior portion of the eye. A therapeutically effective amount of the composition of this presently claimed and disclosed inventive concept(s) can be delivered to the choroid and retina proximal to the macula of the eye to retard the growth of blood vessels that lead to macular degeneration in the eye and/or to retard retinal cell degeneration.

In one aspect, therapeutic compositions of this presently claimed and disclosed inventive concept(s) can be administered to the eye or other areas of the body by a number of techniques including by use of medical devices and methods of administration known in the art, such as for example those described in U.S. Pat. Nos. 6,397,849; 6,299,895; 5,770,589; 5,767,079; 5,707,643; 5,632,984; 5,443,505; 5,399,163; 5,383,851; 5,273,530; 5,064,413; 4,941,880; 4,790,824; 4,596,556; 4,487,603; 4,486,194; 4,475,196; 4,447,224; 4,447,233; and 4,439,196, each of which is expressly incorporated herein by reference in its entirety. Many other methods of administration such as a single or multiple implant and/or biodegradable matrix composition for controlled release the nanoceria of this presently claimed and disclosed inventive concept(s), an implantable hydrogel matrix which can be biodegradable, an injectable delivery system such as a liposome suspension, injection methods such as comprising a needle less syringe or cannula or needle and syringe, poorly water soluble and biodegradable carriers, and delivery routes that are applicable to administer a drug to the eye and to blood vessels that feed blood to the eye can be used with the compositions of this presently claimed and disclosed inventive concept(s).

For example, the presently claimed and disclosed inventive concept(s) can be delivered by a variety of techniques to the macula region of the eye, preferably to the posterior segment of the eye proximal to the macula. Examples of such techniques include: (a) use of a sterile, pharmaceutically acceptable biodegradable scleral plug which comprises nanoceria and optionally a pharmaceutically acceptable biodegradable matrix such as a polylactic acid or polyglycolic acid or a copolymer of lactic acid and glycolic acid, which plug can be inserted into the eye via an incision in the sclera; (b) use of an implant comprising nanoceria of this presently claimed and disclosed inventive concept(s) and optionally a pharmaceutically acceptable biodegradable matrix wherein the sclera is cut to expose the suprachoroid and wherein the implant is placed into a suprachoroidal space from which implant the nanoceria are released, for example, into the vitreous region of the eye; (c) use of intravitreal injection into the vitreous body of a pharmaceutical composition comprising nanoceria and a sterile aqueous carrier; (d) injection or infusion via a flexible cannula that can be inserted through the posterior sclera and down into the subretinal space at the posterior region of the eye; and (e) by injection of a pharmaceutical composition comprising nanoceria and a pharmaceutically acceptable carrier into an avascular region of the sclera to form a depot comprising nanoceria within the scleral layer and from which the nanoceria can diffuse to the macula, choroid layer, and/or retina.

In one aspect, a pharmaceutical composition used in the presently claimed and disclosed inventive concept(s) can comprise a pharmaceutically acceptable carrier selected from the group consisting of poly(ethylene-co-vinyl acetate), PVA, partially hydrolyzed poly(ethylene-co-vinyl acetate), poly(ethylene-co-vinyl acetate-co-vinyl alcohol), a cross-linked poly(ethylene-co-vinyl acetate), a cross-linked partially hydrolyzed poly(ethylene-co-vinyl acetate), a cross-linked poly(ethylene-co-vinyl acetate-co-vinyl alcohol), poly-D,L-lactic acid, poly-L-lactic acid, polyglycolic acid, PGA, copolymers of lactic acid and glycolic acid, polycaprolactone, polyvalerolactone, poly (anhydrides), copolymers of polycaprolactone with polyethylene glycol, copolymers of polylactic acid with polyethylene glycol, polyethylene glycol; fibrin, Gelfoam™ (which is a water-insoluble, off-white, nonelastic, porous, pliable gel foam prepared from purified gelatin and water for injection), and combinations and blends thereof. Copolymers can comprise from about 1% to about 99% by weight of a first monomer unit such as ethylene oxide and from 99% to about 1% by weight of a second monomer unit such as propylene oxide. Blends of a first polymer such as gelatin and a second polymer such as poly-L-lactic acid or polyglycolic acid can comprise from about 1% to about 99% by weight of the first polymer and from about 99% to about 1% of the second polymer.

The term “pharmaceutically acceptable carrier” or “adjuvant” and “physiologically acceptable vehicle” and the like are to be understood as referring to an acceptable carrier or adjuvant that may be administered to a subject, together with nanoceria of this presently claimed and disclosed inventive concept(s). Further, as used herein “pharmaceutically acceptable carrier” or “pharmaceutical carrier” are known in the art and include, but are not limited to, 0.01-0.1 M solutions and preferably 0.05 M phosphate buffer, or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

In addition, the term “pharmaceutically effective amount” or “therapeutically effective amount” refers to an amount (a dose) of nanoceria effective in treating a patient, having, for example, a site of neovascularization or retinal degeneration. It is also to be understood herein that a “pharmaceutically effective amount” may be interpreted as giving a desired therapeutic effect, either taken in one dose or in any dosage or route or taken alone or in combination with other therapeutic agents.

Advanced wet AMD is a disease of the eye which comprises neovascularization of the choroid tissue underlying the photoreceptor cells in the macula. As noted above, AMD, particularly in its advanced stages, is characterized by the pathological growth of new blood vessels in the choroid underlying the macula. Angiogenic blood vessels in the subretinal choroid can leak vision obscuring fluids, leading to blindness.

In one aspect, diseases of the eye which exhibit neovascularization proximal to the retina such as wet AMD, retinitis pigmentosa, Stargardt's Disease, diabetic retinopathy, hypertensive retinopathy, and occlusive retinopathy can be treated to reduce the rate of neovascularization by administration of a composition of this presently claimed and disclosed inventive concept(s) comprising nanoceria having angiogenesis inhibiting activity.

The compositions of the presently claimed and disclosed inventive concept(s) when administered to the eye or to blood vessels that feed into the eye of a patient can be useful to treat ocular diseases such as, but not limited to, wet AMD, macular edema, RP, Stargardt's Disease, DR, hypertensive retinopathy, and occlusive retinopathy by reducing the rate of formation of neovascularization and thereby slow the progress of the disease. The rate of neovascularization which occurs in such a disease in a patient is preferably reduced by administration of the nanoceria of this presently claimed and disclosed inventive concept(s) to 90%, more preferably to 50%, even more preferably to 25%, even more preferably to 10%, even more preferably to 5%, even more preferably to 1%, and most preferably to 0.1% or less of the rate of neovascularization which occurs in such a disease in the absence of administration of the nanoceria of this presently claimed and disclosed inventive concept(s) (i.e., in an untreated patient).

Administration of a pharmaceutical composition comprising the nanoceria of this presently claimed and disclosed inventive concept(s) to a subject in need of treatment for a disease of the eye such as macular degeneration, retinitis pigmentosa, Stargardt's Disease, diabetic retinopathy, hypertensive retinopathy, and occlusive retinopathy can substantially reduce or prevent angiogenesis associated with subretinal neovascularization, choroid neovascularization underlying the macula, and a proliferation of neovascular tissue in the subretinal choroid proximal to the macula in an eye in a mammalian subject and can substantially reduce or prevent retinal degeneration associated with such pathologies of the eye.

The method can be useful as a prophylactic treatment to prevent further onset or progression of macular or retinal degeneration (e.g., due to rod and cone photoreceptor cell death) in an eye that exhibits symptoms of a disease of the eye such as macular degeneration, retinitis pigmentosa, Stargardt's Disease, diabetic retinopathy, hypertensive retinopathy, and occlusive retinopathy. In another aspect, the method can be useful as a prophylactic treatment to prevent the deposition of druzen and the death of photoreceptor cells in the macula or elsewhere in the retina. In one aspect of the presently claimed and disclosed inventive concept(s), the method can prevent the death of photoreceptor cells (which photoreceptor cells are also herein referred to as photoreceptors) in the eye of a subject by acting on intracellular mechanisms of the regulation of cell death. The method can also be useful to prevent onset or progression of macular degeneration in an eye that does not exhibit vision-obscuring symptoms of macular degeneration, especially in an eye of a patient whose other eye does exhibit vision-obscuring symptoms of macular degeneration.

In another aspect of this presently claimed and disclosed inventive concept(s), a method of treatment of a disease of the eye such as macular degeneration, retinitis pigmentosa, Stargardt's Disease, diabetic retinopathy, hypertensive retinopathy, and occlusive retinopathy comprises administration such as by injection or implantation (or other method described herein) into tissue proximal to the eye of a therapeutically effective amount of nanoceria of this presently claimed and disclosed inventive concept(s), or of a sterile pharmaceutical composition of this presently claimed and disclosed inventive concept(s) suitable for injectable administration and comprising nanoceria of this presently claimed and disclosed inventive concept(s) and a carrier suitable for injectable use (e.g., sterile, sterilizable, and isotonic with blood), which can prevent or delay the onset of retinal cell degeneration or of angiogenesis associated with subretinal neovascularization, choroid neovascularization underlying the macula, and a proliferation of neovascular tissue in the subretinal choroid proximal to the macula in an eye of a patient.

The presently claimed and disclosed inventive concept(s) is, in one embodiment, a method of inhibiting, reducing or reversing the rate of retinal cell degeneration, especially of rod and cone photoreceptor cells, in a mammalian subject in need of such treatment, wherein the method comprises administering to the mammalian subject a therapeutically effective amount of a composition comprising cerium oxide nanoparticles, thereby inhibiting, reducing, or reversing the rate of retinal cell degeneration and in the mammalian subject. The subject may have an ocular condition which is, for example, at least one of age-related macular degeneration, diabetic retinopathy, retinal angiomatomous proliferation, glaucoma, retinitis pigmentosa, retinal detachment, inherited retinal degeneration, Stargardt's disease, hypertensive retinopathy, and occlusive retinopathy.

The presently claimed and disclosed inventive concept(s) further comprises a combination therapy wherein a VEGF inhibitor may be administered in combination with the present nanoceria therapy. The nanoceria may be administered with a cytotoxic agent, a chemotherapeutic agent or a growth inhibitory agent can be administered to the subject together in a single dosage composition such as a combined formulation, or each agent can be administered in a separate dosage formulation. Where separate dosage formulations are used, the nanoceria of the presently claimed and disclosed inventive concept(s) and one or more additional agents can be administered concurrently, or at separately staggered times, i.e., sequentially. The therapeutic methods of the presently claimed and disclosed inventive concept(s) may also be combined with other agents or medical procedures used for treatment of eye disorders or other pathological conditions expressing neovascularization.

Nanoceria particles are contemplated herein for use in preventing blindness due to a variety of diseases including, but not limited to, macular degeneration, hereditary retinitis pigmentosa, glaucoma, diabetes, and retinal detachment.

Investigations of nanocrystalline cerium oxide (e.g., CeO₂ and Ce₂O₃) nanoparticles (nanoceria) have revealed that its lattice constant increases with decreasing nanoparticle size. This has been attributed to an increase in oxygen vacancies in the crystal structure. This suggests that the migration enthalpy of the oxygen vacancy in CeO₂ is smaller at the nanoscale. Additionally, at the nanoscale, the surface area of CeO₂ particles is dramatically enlarged in relation to its volume which increases oxygen exchange and redox reactions. Thus, oxygen vacancies are likely to form more readily at the nanoscale.

It is believed, without wishing to be bound by theory, that nanoceria, owing to their chemical and physical structure, can protect cells from ROS or free-radical-induced damage. This is especially supported by the demonstration that nanoceria have catalytic activities like those of two major anti-oxidative enzymes, super oxide dismutate and catalase. The “defects” in the nanoceria particles can act as chemical spin traps similar to nitrosone compounds, currently used as biological antioxidants. It is believed that the nanoceria act as free-radical scavengers by switching between the +3 and +4 valence states via various surface chemical reactions and one CeO₂ nanoparticle may offer many sites of spin-trap activity, whereas current pharmacological agents offer only a few per molecule. Additionally, the lattice defects in nanoceria can regenerate and do not necessarily require repetitive dosages as seen with the use of dietary supplements of antioxidants such as vitamins C and E. It was previously demonstrated that the intravitreal injection of nanoceria, which catalytically scavenge ROS, prevents light damage and blindness in albino rats. Herein we demonstrate (as described below) that nanoceria catalytically destroy ROS in the retinas of VLDLr Knock Out mice which in turn prevent oxidative stress and ROS induced damage such as increases in VEGF, choroidal neovascularization, retinal vascular lesions, degeneration of photoreceptor cells and the subsequent loss of vision.

### EXAMPLES

While the presently claimed and disclosed inventive concept(s) will now be described in connection with certain preferred embodiments in the following examples so that aspects thereof may be more fully understood and appreciated, it is not intended to limit the presently claimed and disclosed inventive concept(s) to these particular embodiments. On the contrary, it is intended to cover all alternatives, modifications and equivalents as may be included within the scope of the presently claimed and disclosed inventive concept(s) as defined by the appended claims. Thus, the following examples, which include preferred embodiments will serve to illustrate the practice of this presently claimed and disclosed inventive concept(s), it being understood that the particulars shown are by way of example and for purposes of illustrative discussion of preferred embodiments of the presently claimed and disclosed inventive concept(s) only and are presented in the cause of providing what is believed to be the most useful and readily understood description of formulation procedures as well as of the principles and conceptual aspects of the presently claimed and disclosed inventive concept(s).

The VLDLr KO Mouse Model.

The Very Low Density Lipid Receptor Knock Out mouse (VLDLr KO), (see Heckenlively et al., “Mouse model of subretinal neovascularization with choroidal anastomosis”. Retina 2003; 23: 518-522), is an outstanding animal model for AMD and in particular for RAP (Retinal Angiomatous Proliferation) in which there is a progressive increase in retinal VEGF and VEGF-induced vascular proliferation within the photoreceptor cell outer nuclear layer as well as an accumulation of lipid particles with degeneration of Bruch's membrane. This mouse also exhibits subretinal choroidal neovascularization. Oxidative stress in the VLDLr KO mouse has also been shown to be associated with a low antioxidant capacity in its mitochondria. The overall phenotype is very similar to that associated with a subset of AMD patients who have Retina Angiomatous Proliferation (RAP). VEGF is responsible for angiogenesis and vasculature formation in the eye and numerous strategies have been designed to inhibit its activity in many diseases, including AMD and cancer. Currently, the only effective therapy for wet AMD is the intravitreal injection, every 4-6 weeks, of monoclonal antibodies against VEGF, aptamers which inhibit VEGF activity or soluble receptors for VEGF which act as “VEGF traps”. Our recently published data (Li et al., “Biochemical alterations in the retinas of very low-density lipoprotein receptor knockout mice: an animal model of retinal angiomatous proliferation”. Arch Ophthalmol 2007; 125: 795-803), demonstrated the rise in VEGF in the ONL of the retina, the developmental increase in illicit blood vessels and the increase in pro-inflammatory enzymes prior to vascularization. We hypothesized herein that nanoceria, through scavenging of ROS, can inhibit the rise in VEGF, the subsequent angiogenesis and vascular lesions in the retina and the eventual photoreceptor cell death. Data provided herewith support this assertion.

In this mouse model (VLDLr KO) for macular degeneration, blood vessels form and grow from the retinal vasculature through the choroid and retinal pigment epithelium layer. In some forms of macular degeneration and in diabetic retinopathy, the retinal blood vessels also become “leaky”. As shown in the data below, administration of nanoceria particles prevents both choroidal neovascularization (CNV) and leakage of the retinal blood vessels.

We hypothesized that the nanoceria, by a mechanism involving the elimination of endogenously produced ROS, inhibit the mutant phenotype of the VLDLr−/−mouse. The phenotypic characteristics which were measured were: ROS and ROS-mediated damage in the retina; the illicit rise in Vascular Endothelial Growth Factor (VEGF); retinal vascular lesions; subretinal neovascularization; disruption of Bruch's membrane and the retinal pigment epithelium cell layer by choroidal neovascular tufts. Quantitative histology, using bright field microscopy on hematoxylin and eosin (H&E) stained retinal sections, were used to evaluate the morphological preservation of photoreceptor cells whereas retinal function was determined using electroretinography. VEGF levels in the retina were assayed with anti-VEGF immunoblots, ELISAs and immunocytochemistry whereas its mRNA were assayed by RT-PCR. Superoxide radicals in the retina were assessed using a hydroxyethidine assay whereas H₂O₂ were assayed with 2′,7′-dichloro-dihydro-fluorescein-diacetate. ROS-induced damage was visualized with antibodies against products of ROS activity including acrolein, nitrotyrosine and 8-hydroxy-2-deoxy-guanosine. The effects of the nanoceria on neuroprotective pathways were analyzed by Western blots and cDNA micro arrays. We have established a vascular filling assay to visualize both the retinal vasculature and choroidal neovascularizations. Our new Olympus Zoom Fluorescence Microscope (MVX10) for macro to microfluorescence imaging enabled us to perform fluorescein angiography on the same live mouse and follow the development and/or regression of the choroidal neovascular tufts and the retinal vascular blebs and lesions on successive days for the same mouse. A major advantage of our current in vivo assays is that the data can be obtained within one week.

### Example 1

Effects on ROS and ROS-Induced Damage.

We first performed experiments to determine if there actually was an increase in ROS in the retina of the VLDLr KO mouse and if the nanoceria could reduce the ROS and the damage caused by them. In a cell, the three major sources of intracellular ROS are from 1) mitochondrial oxidative respiration, 2) NADPH-oxidase and 3) Nitrous oxide synthetase (NOS3).

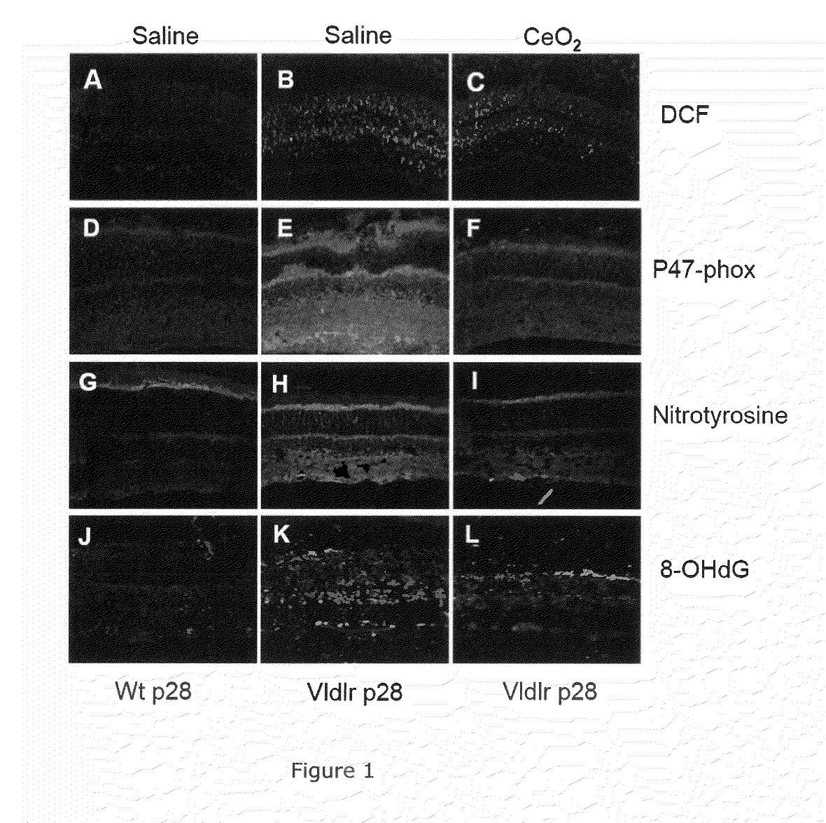

The 2′,7′-dichloro-dihydro-fluorescein-diacetate (DCF) assay for detection of cellular oxidation by hydrogen peroxide, peroxynitrite and hydroxyl radicals was used with cryostat sections of eyes from normal and VLDLr−/−mice which had been injected intravitreally with either saline or nanoceria on post-natal day 7 (P7) and assayed on post-natal day 28 (P28). Fluorescence microscopy of cryostat sections from 28 day mice (FIG. 1A) detects some DCF signal in the wild type retina but very significant amounts in the VLDLr−/−retina (FIG. 1B). However, injection of the nanoceria on P7 greatly reduces the presence of ROS in the VLDLr−/−retina on day 28 (FIG. 1C). Another major contributor to the intracellular production of ROS is NADPH Oxidase. The immunocytochemical staining for the activating subunit of NADPH Oxidase (P-47) reveals (FIG. 1D) its presence throughout the normal retina. The VLDLr−/−retina shows more intense staining of all retinal layers (FIG. 1E) whereas the intensity of staining in the retina of the nanoceria-injected VLDLr−/−mouse was reduced almost to the control level (FIG. 1F).

As levels of nitrous oxide species rise within the cell, the nitrosylation of tyrosine residues in proteins increases. To determine if there was an increase in the nitrosylation in the VLDLr−/−retina, immunofluorescence microscopy was performed using anti-nitrotyrosine immunoglobulins and fluorescein conjugated secondary antibodies. The results show that there is a detectable staining of the wild type retina (FIG. 1G) and a very intense staining of the VLDLr−/−retina (FIG. 1H) which is decreased to the control level in the nanoceria injected (FIG. 1I) VLDLr−/−mouse. Oxidative damage to DNA results in the production of 8-hydroxydeoxyguanisine which can also be detected by immunocytochemistry. Representative results from such an assay show that the control retina (FIG. 1J) has detectable levels of OHdG and the VLDLr−/−retina (FIG. 1K) has much higher levels. Treatment with the nanoceria reduces the amount of OHdG in the VLDLr−/−retina (FIG. 1L) to that which is detected in the wild type control. Each of these four independent assays shows an increase in oxidative stress in the VLDLr−/−retina compared to the wild type control and demonstrate a decrease in oxidative stress in the retinas of VLDLr−/−mice injected with nanoceria. Collectively, these data indicate that oxidative damage increases in the mouse retina as a result of the knockout of the VLDLr−/−gene and that the presence of nanoceria inhibits those increases.

### Example 2

VEGF—Western Blot Data.

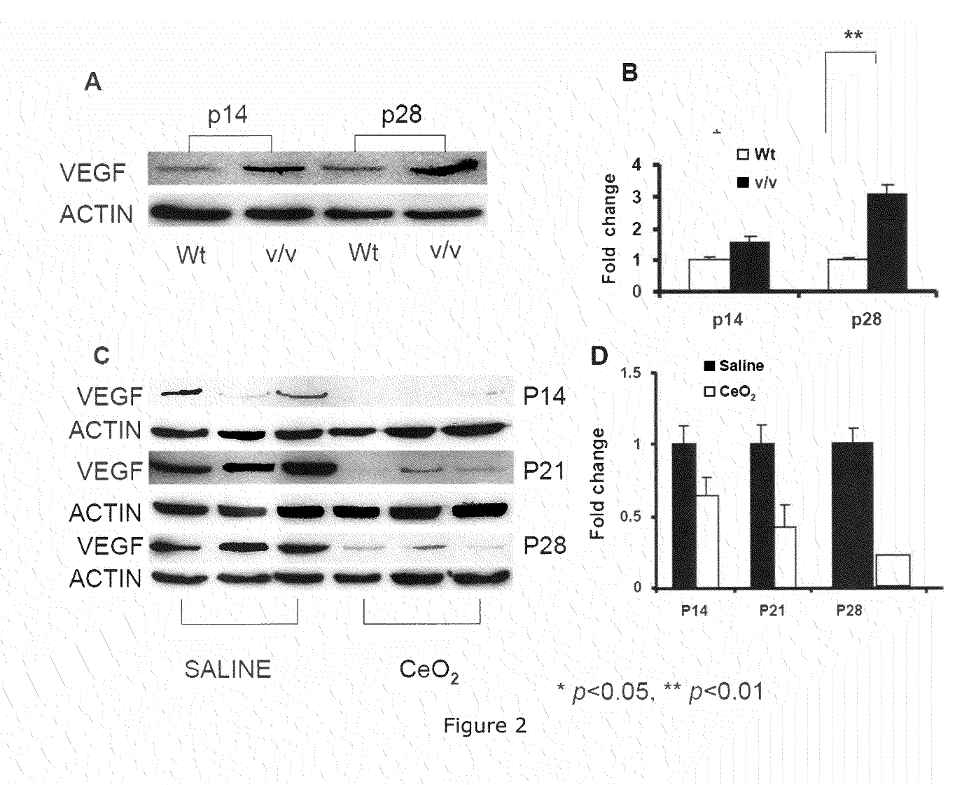

Mice were injected intravitreally with either 1 μl of PBS or 1 ul of PBS plus nanoceria at post-natal day 7P7 and the animals killed on P14, P21 or P28. Retinas were homogenized, subjected to SDS-PAGE, and blotted to nitrocellulose. The bands were detected with primary and secondary antibodies and visualized with an HRP-DAB assay (FIG. 2). Wild type (+/+) retinas had barely detectable levels of VEGF with or without nanoceria. The VLDLr KO retinas had about an eightfold increase in VEGF compared to the +/+ retinas. However, the nanoceria (“nanoparticle”) injected VLDLr KO mice had about 60% less VEGF at day 21. These data indicate that VEGF increased in the VLDLr KO retinas due to ROS and that it decreased because of the nanoceria mediated decrease in ROS.

The amount of VEGF, as determined by Western blots, is higher in the VLDLr−/−retina than in the wild type retina even at P14 and progressively increases (FIG. 2A). Densitometry (FIG. 2B) indicates that by P28 VEGF is at least 3 fold higher than in control retinas. Injection of nanoceria at P7 results in a progressive decrease in VEGF levels in the VLDLr−/−retina such that by P28, the amount is about 5 fold less than that in the saline injected eyes (FIG. 2C,D). These data are consistent with the interpretation that the scavenging of ROS and the inhibition of oxidative damage by the nanoceria inhibit “downstream”events.

### Example 3

VEGF—Localization by Immunofluorescence.

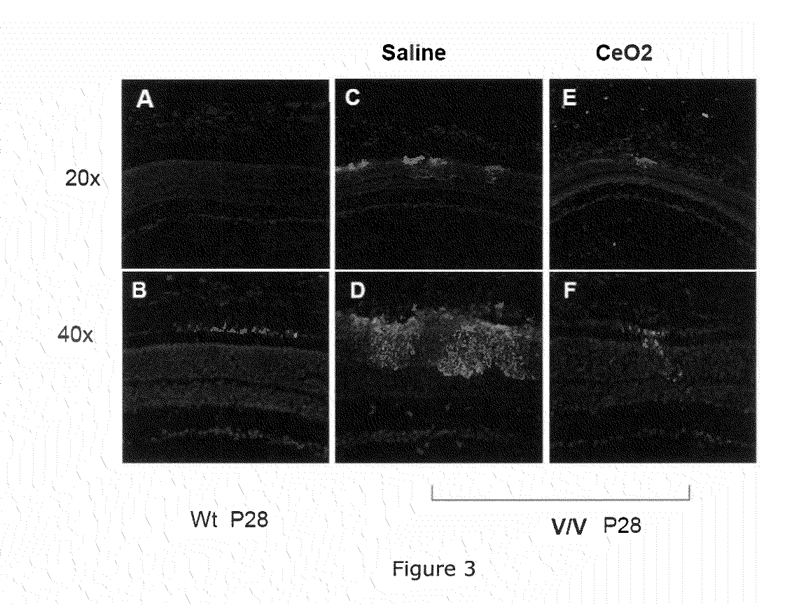

To determine where VEGF was localized in the wild type and the VLDLr KO retinas and whether the nanoceria had any effect on the localization, Alexa green-conjugated secondary antibodies were used in combination with anti-VEGF primary immunoglobulins. The wild type mouse retinas (C57BL/6J) (FIG. 3A,B) had very little VEGF and it was localized to the outer segments of the retina. However, the pattern of labeling with anti-VEGF in the VLDLr KO retina (FIG. 3C,D) was heavy but discontinuous; predominantly in the outer and inner segments of photoreceptors; and especially in their perinuclear regions in the ONL adjacent to vascular lesions. The intensity of labeling progressively diminished as the distance from the lesion increased. The age-matched VLDLr mice, which had received an intravitreal injection (1 ul of 1 mM) of nanoceria on P7, had fewer vascular lesions and exhibited greatly reduced staining surrounding the remaining lesions (FIG. 3E-F). Individual “optical sections” reveal uniform labeling of the cytoplasm surrounding the photoreceptor cell nuclei and inner segments which suggests that the VLDLr KO photoreceptor cells are making VEGF. Also, a single injection of the nanoceria at P7 inhibits the developmental increase in retinal VEGF for at least three weeks. These data demonstrate that the rise in retinal VEGF in the VLDLr KO retina is due to ROS and can be prevented by the scavenging activities of nanoceria.

### Example 4

Nanoceria inhibit development of “neo” leaky retinal vasculature in VLDLr KO retinas.

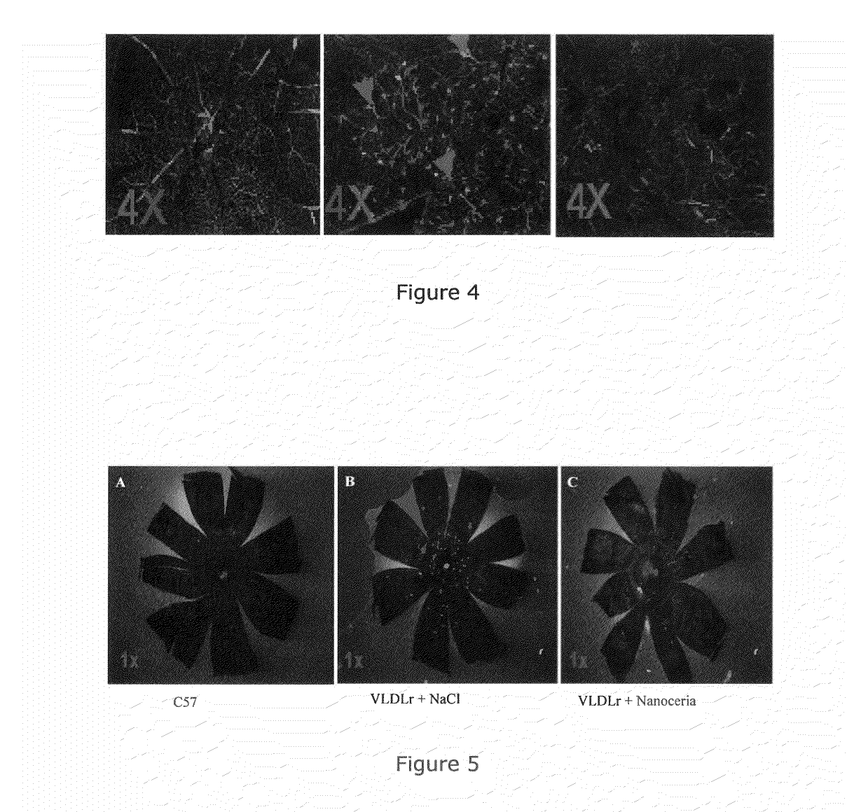

The retina has two blood supplies, the retinal vasculature and the choroid. The VLDLr KO retina has problems with both systems. The retinal vasculature of the VLDLr KO mouse exhibits a developmental increase in “neo” vessels which are absent from control retinas suggesting the possibility that these vessels arise as a result of the increase in retinal VEGF and that the nanoceria can inhibit their development. To test this, we used fluorescein-conjugated Dextran to visualize the retinal vasculature and asked if an intravitreal injection of the nanoceria on P7 could inhibit the formation of these illicit blood vessels when visualized on P28. Representative images from such an experiment are shown in FIG. 4. With this assay, the larger vessels in the control C57 retinas (FIG. 4A) are very bright with the smaller vessels forming a less intensely labeled meshwork. Injection of nanoceria or saline has no effect on the normal retinal vasculature so that data is not shown. The VLDLr KO retinal vessels of saline injected (FIG. 4B) or uninjected (not shown) mice have brightly labeled newly formed vessels with coiled ends (appearing as “blebs”) which project toward the RPE cells. The nanoceria injected VLDLr KO eyes (FIG. 4C) show greatly reduced numbers of these neovessels.

### Example 5

Nanoceria inhibit development of choroidal neovascular tufts.

The vascular filling assay also enables choroidal neovascular connections between the choroid and retina blood supplies to be visualized. In this case, after the retina has been removed the remaining RPE-sclera-choroid is “pie-cut” to allow flat mounting and placed with the RPE facing up (FIG. 5). The pigmented RPE prevent visualization of any of the choroidal vasculature in the normal C57 mouse (FIG. 5A) but the eyecup from the saline injected VLDLr KO mice (FIG. 5B) had numerous bright choroidal neovascular tufts which projected through the RPE cell layer. However, the eyes of the VLDLr KO mice which had been injected with nanoceria (FIG. 5C) have far fewer choroidal neovascular tufts.

### Example 6

Vascular Lesions Can Be Quantified.

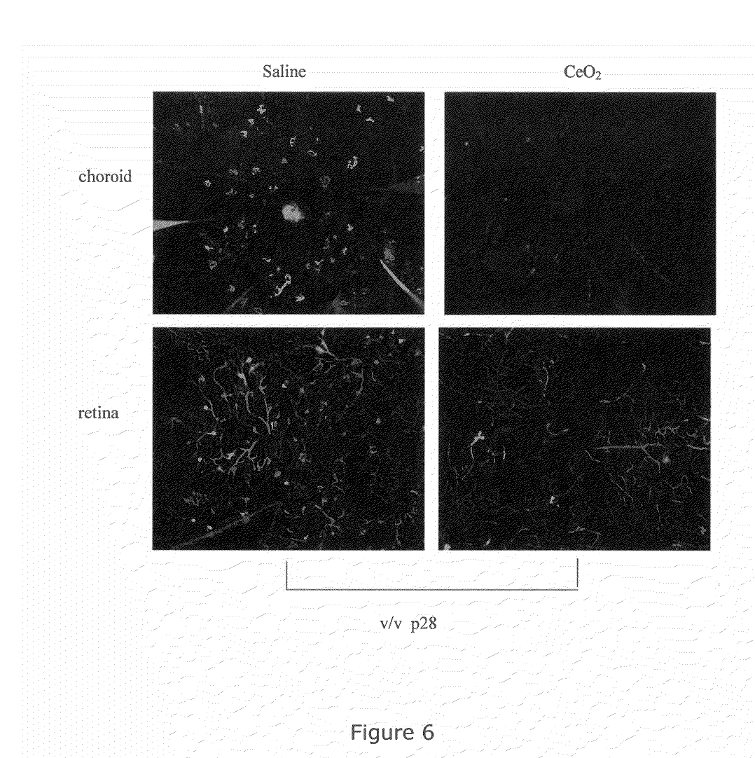

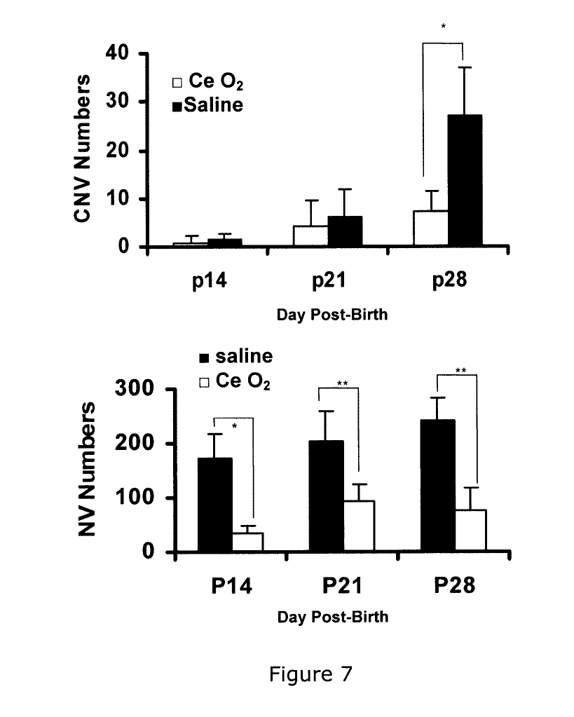

Because choroidal neovascular “tufts” and retinal neovascular “blebs” are readily visible, both can be quantified. The results of such an experiment are presented in FIG. 6 and FIG. 7 and demonstrate that the number of choroidal tufts present during development increases, especially by P28. The data also show that a single injection of nanoceria at P7 decreases the number of such tufts seen in P14, P21 and P28 VLDLr KO choroids. The confocal microscope shows the vascular lesions as “blebs” which are present in significant numbers even at P14 and progressively increase at P21 and P28. As with the tufts, the developmental appearance of the blebs is strongly inhibited by a single injection of nanoceria at P7. These data strongly support our hypothesis that ROS represents an important connection between the primary defect and the downstream effects which in the VLDLr KO mouse are vascular defects. These data also indicate that by counting blebs and tufts, the effectiveness of a therapeutic treatment can be evaluated. Therefore, dose response experiments can be done to determine the lowest dose of nanoceria that is most effective. This assay also enables a comparative analysis to be performed between nanoceria and other antioxidant agents. Collectively, these data demonstrate that the illicit angiogenesis in the VLDLr−/−eye can be inhibited by an injection of the nanoceria and that the extent of inhibition can be quantitated.

### Example 7

Regression of Vascular Anomalies by Treatment with Nanoceria.

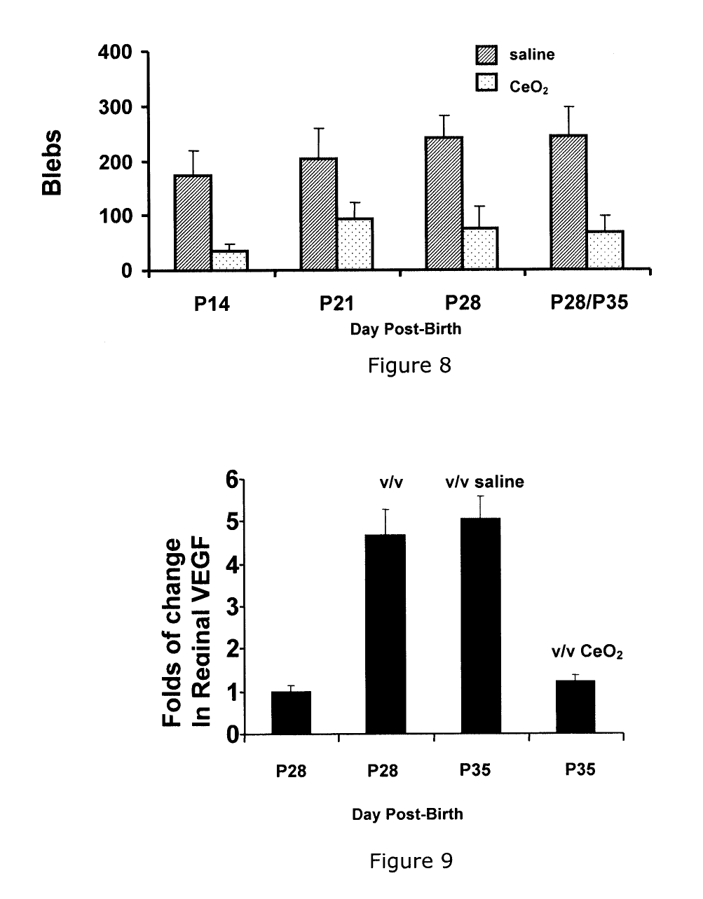

Although these data were scientifically very important for our hypothesis, from a therapeutic perspective, the nanoceria would be much more useful if they could decrease or eliminate the vascular anomalies which were present prior to their injection. To examine this possibility, we added an experimental paradigm to that which is seen previously in FIG. 6. We also asked what would happen to the retinal blebs already present prior to injection. To answer this, mice were injected on day P28 and the vascular filling assay was done on day P35. The P28/P35 data are shown in FIG. 8 and indicate that the retinal vascular lesions, blebs, have slightly increased in the saline injected mice but in the nanoceria injected mice the blebs have regressed to the same level as was found on P28 when the mice were injected at P7. Similar data was obtained when the CNV blobs were counted (data not shown). These are especially important observations because they indicate that the vascular blebs and blobs present at the time of injection on day P28 are dependent on the presence of ROS and their downstream effects. Therapeutically this means the nanoceria are effective in subjects which already have vascular anomalies. These data also indicate that the continued presence of these illicit new blood vessels in the retinal vasculature requires the continual production of ROS and that the “assay” for therapeutic effects can be done within a week.

In a separate parallel experiment, VEGF was quantified using densitometry of anti-VEGF bands on immunoblots. The data (FIG. 9) show that the injection of nanoceria at P28 into a mature mouse decreases VEGF by P35 to the amount of retinal VEGF found in normal retinas.

### Example 8

Illicit blood vessels in the VLDLr−/−retina grow from the ONL into the choroid subretinal space.

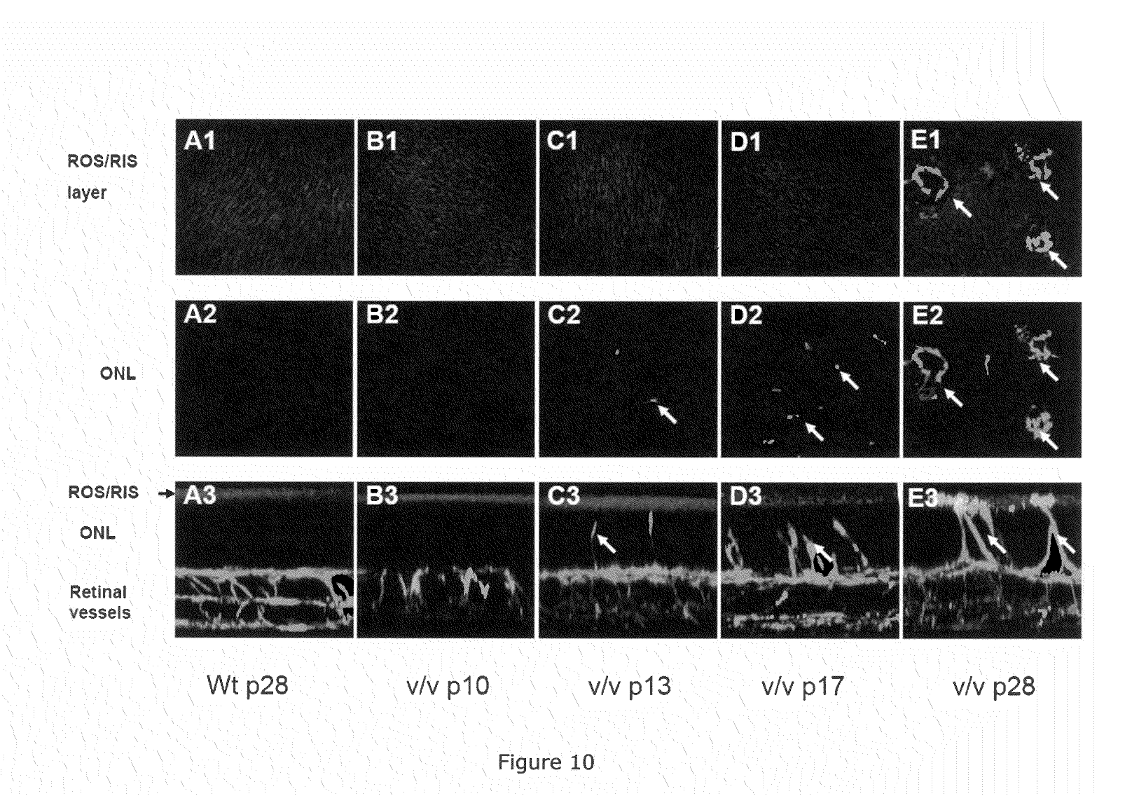

Retinal angiomatous proliferation in humans has been described as having the neovessels growing from the existing vessels in the neural retina through the ONL towards the choroid where they eventually puncture the RPE and fuse with the choroidal blood supply. The direction of vascular growth in the VLDLr−/−retina was indicated to be from the ONL into the choroid (Heckenlively, 2003, 9846; Hu, 2008, 9936) but this is disputed in a recent paper in which it was concluded that the vessels actually originate in the choroid (Wu, 2008, 9937). The application of confocal microscopy, the vascular filling assay and a developmental study of the VLDLr−/−retina has allowed us to distinguish between these alternative conclusions. Following the labeling of the vascular system with fluorescein (green) conjugated Dextran, whole mounts of the VLDLr−/−retinas were incubated with Alexa Red conjugated-peanut agglutinin to label the sheaths surrounding the outer segments of cones. The retina was then mounted with the photoreceptors facing up and the ganglion cell layer down. Optical sections were taken every 0.5 μm from the top down. This enabled defined stacks of sections to be visualized as well as the 3-Dimensional stack through the entire retina to be rotated 90 degrees. Representative images from such studies are shown in FIG. 10. The cone sheaths form a continuous surface (FIG. 10, A1-E1) in the P28 wild type C57 retina and in VLDLr−/−retinas at P10, P13 and P17. However at P28, this continuity is disrupted by blood vessels which result in areas immediately adjacent to the protruding vessels being devoid of cones. Looking down at the stacked images of the region of the ONL (FIG. 10, A2-E2) shows no vessels in the P28 C57 ONL nor in the P10 VLDLr−/−ONL. Vessels first appear as dots in the VLDLr−/−P13 ONL (FIG. 10, C2) and progressively increase in number through P17 (FIG. 10, D2) and then coalesce by P28 (FIG. 10, E2). Using animation to rotate the entire stack of optical sections (FIGS. 10, A3-E3) 90°, a cross-section is seen in which the cones are at the top. In the P13 VLDLr−/−retina (FIG. 10, C3) neovessels can be seen to grow from the outer plexiform region into the ONL in increasing numbers (FIG. 10. D3) until they coalesce and push through the cone outer segments (FIG. 10. E3) to fuse with the choroidal neovascular vessels. These data definitively demonstrate that the neovessels in the VLDLr−/−mouse grow from the retina towards the RPE and choroid and not in the reverse direction. These data are also consistent with the high concentration of VEGF in the ONL of the VLDLr−/−retina.

### Example 9

Nanoceria Cause Regression of Existing Illicit Neovessels.

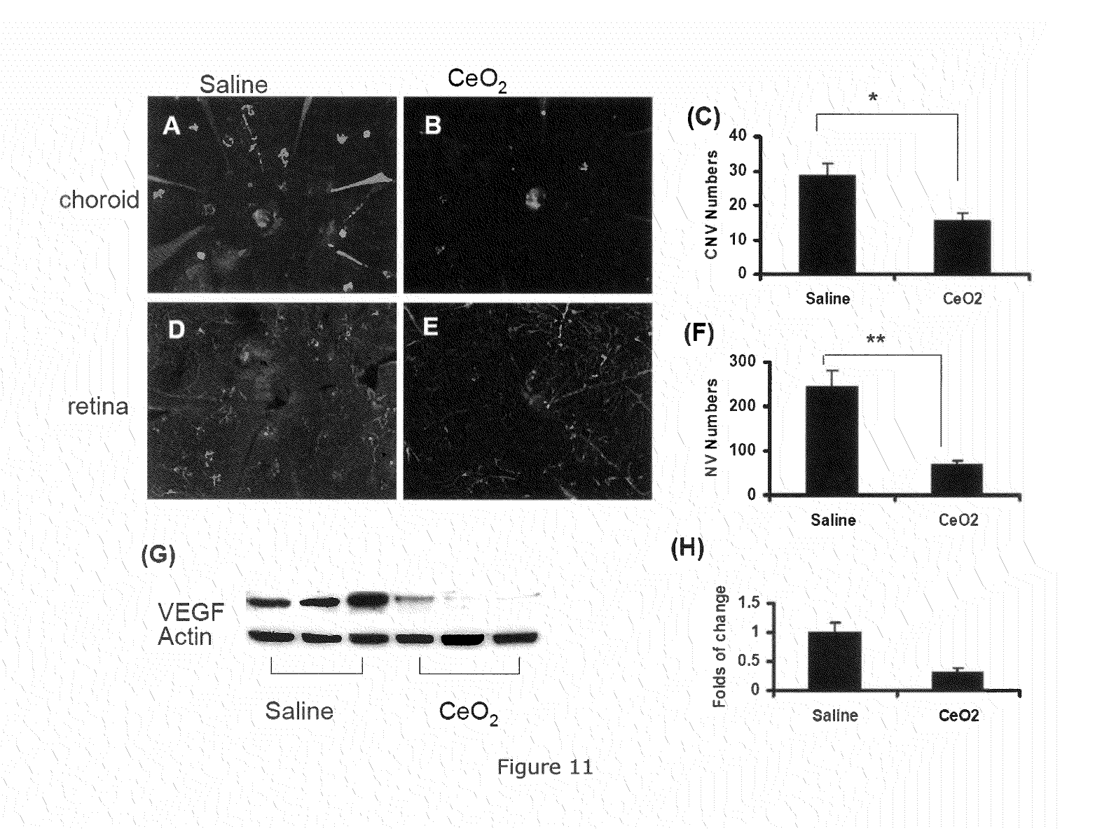

Injection of nanoceria at P7 was demonstrated to prevent the developmental increase in VEGF (FIG. 2) and in the retinal vascular blebs and choroidal neovascular tufts (FIG. 6) when analyzed at P28. We next asked whether developmental changes which had occurred prior to injection of nanoceria could be reversed. For this experimental paradigm (FIG. 11), VLDLr−/−mice received an intravitreal injection of nanoceria at P28 and the eyes were analyzed a week later on P35. These data are striking in that the presence of the nanoceria for only one week caused down regulation of VEGF (FIG. 11G,H), regression of choroidal neovascular tufts (FIG. 8A,B,C) and retinal vascular lesions (blebs) (FIG. 11D,E,F) to levels similar to those which resulted from the presence of nanoceria for the three week period from P7 to P28. We conclude from these data that the continued presence of elevated levels of VEGF and the illicit neovessels in the retina and the choroid, require the continual production of ROS and that their inhibition by nanoceria produces a rapid decrease in each parameter.

The results provided hereinabove establish a direct link between oxidative stress and retinal neovascularization in the inherited VLDLr−/−mouse model. Without wishing to be bound by theory, the results indicate that the retinal vascular defects in VLDLr−/−mice are primarily due to oxidative stress. When the ROS level is lowered by a single injection of nanoceria in the vitreous during early postnatal development, a concomitant reduction of the angiogenic factor VEGF in the ONL is observed, and an inhibition of the development of intra- and subretinal neovascularization (NV). It is further shown herein that the maintenance of new blood vessels relies on sustained oxidative stress because when the ROS level is reduced by a single injection of nanoceria at P28, within one week, we observe a regression of these vascular defects.

These results support a causative relationship between oxidative stress and new blood vessel formation in the VLDLr−/−retinas and indicate that the antioxidant nanoceria will make an effective therapeutic agent to combat ocular neovascular diseases.

The nanoceria used herein have prolonged antioxidant and anti-angiogenic effects. In this work it is shown that the nanoceria possess regenerative radical scavenging activity in vivo. The level of ROS measured by DCF fluorescence remains at reduced level three weeks after nanoceria application. Besides ROS reduction, we observed significant reduction of intra- and subretinal NV using this experimental paradigm when we inject nanoceria at P7 and analyze the anti-angiogenic effect at P28. This prolonged anti-angiogenic effect exhibited by the nanoceria is a substantial improvement over the transient anti-angiogenic effect offered by the combination treatment of macugen, an integrin antagonist, and T2-TrpRs, a fragment of tryptophan transfer RNA synthetase with angiostatic activity, in the same animal model (Dorrell et al., 2009). The anti-angiogenic effect was observed within 8 days of treatment (P12 injection and P20 analysis). However, the angiostatic effect of the VLDLr KO was abolished after 2-3 weeks of injection. In another experimental paradigm, we administered nanoceria at P28, when retinal NV was well underway, and examined the retinal vasculature one week later at P35. We observed dramatic reductions of VEGF level and intra- and subretinal NV lesions. Without wishing to be bound by theory it appears that oxidative stress lies upstream of the pathogenesis of retinal NV in the VLDLr−/−mice, and that the elimination of oxidative stress prohibits the development and maintenance of new blood vessels.

VEGF Overexpression in the ONL.

Lower levels of VEGF are normally expressed in the rodent retina. We and others (Dorrell et al., 2009) have shown that VEGF is upregulated in the VLDLr−/−retinas very early on (P14) and the level continues to rise with age. It is shown herein that ectopic VEGF in the retina is made by photoreceptor cells and appears to coincide with zones of intra- and subretinal NV. Even though RPE cells exhibit upregulation of VEGF at P14 (Dorrell et al. 2009), retinal degeneration is not seen in VLDLr−/−mice until 3-4 months of age. VLDLr mRNA is highly expressed in the ONL and the outer segment of photoreceptors in the WT retinas (Dorrell et al., 2009). We speculate that the lack of VLDLr causes hypoxic conditions in the ONL, and thus upregulates VEGF expression in the ONL and RPE. Under this condition, endothelial cells from the retinal vasculature sprout new vessels towards the avascular ONL as early as P13.

In summary, it is demonstrated herein that nanoceria are powerful antioxidants that are effective in preventing and abolishing the development and growth of retinal neovascularization in vivo. Also, because preventing these vascular lesions in the VLDLr retina has also been shown to inhibit retinal degeneration, our data suggest that the nanoceria will also prevent the associated degeneration of retinal neurons.

Results with this VLDLr KO mouse model are striking. These data demonstrate that the nanoceria down-regulate retinal VEGF, inhibit the formation of new leaky blood vessels in the retinal vasculature and inhibit choroidal neovascularization. Our assays also enable dose response data to accurately evaluate the effectiveness of different preparations of nanoceria. In addition, because the nanoceria were also shown to be therapeutic even when administered after the illicit retinal vessels have formed, we can now directly compare the efficacy of the nanoceria over time with conventional treatments using anti-VEGF antibodies which are currently injected intravitreally every 4-6 weeks for patients with wet AMD. It is contemplated that nanoceria treatments are effective in ocular conditions such as (but not limited to) macular edema and diabetic retinopathy.

The present data demonstrate four major points. The first is that a single intravitreal injection of nanoceria into the eyes of VLDLr KO mice at P7 prevents the inherited increase in retinal ROS and ROS-mediated damage by a mechanism involving the inhibition of hypoxia. Our data indicate that the three major intracellular sources of ROS which are elevated by hypoxia, (NADPH oxidase, mitochondrial oxidative respiration and nitrous oxide synthase) have decreased production of ROS in the presence of the nanoceria. Secondly, our data show that the nanoceria, by decreasing ROS, also decrease the downstream effects of ROS including: preventing the rise in concentration of retinal VEGF, the development of illicit angiogenesis within the retinal vasculature and the development of subretinal and choroidal neovascularization (CNV). The third major point is that when the nanoceria are injected at P28, all of the disease characteristics which are present at P28 significantly regress (˜80%) within one week. This fact provides us with an assay of relatively short duration (1 week) which enables the effectiveness of nanoceria preparations to be evaluated within one week. Fourth, and especially important regarding the presently claimed and disclosed inventive concept(s), these data demonstrate that VEGF and illicit vessels which are present prior to administration of nanoceria decrease and regress when nanoceria are administered. This indicates that the illicit vessels require ROS. The normal vessels are not affected. This latter point is extremely important with respect to the therapeutic treatment of patients with RAP, AMD, diabetic retinopathy or any other ROS dependent disease because illicit blood vessels in their eyes preferably regress after treatment with the nanoceria without any negative effects on the normal vasculature.

### Example 10

Systemic Injection of Nanoceria.

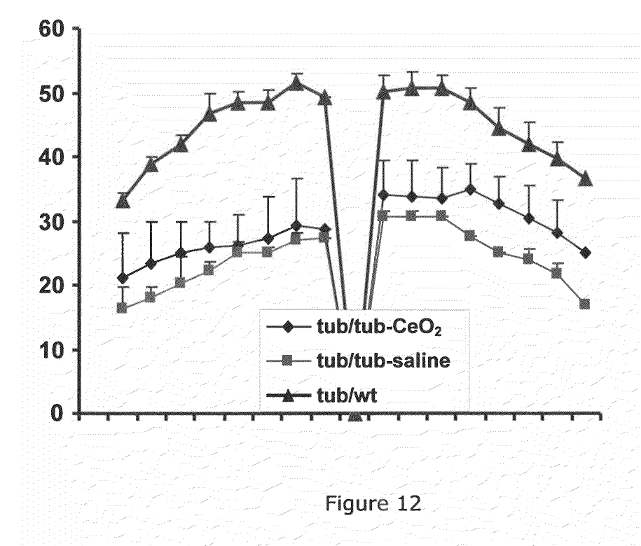

Cerium oxide nanoparticles administered systemically are shown herein to inhibit inherited retinal degeneration in the tubby mouse. To test whether the CNP have a beneficial effect on the tubby photoreceptor cells, mice were injected intravitreally at day P7 with 1 ul of 1 mM CNP (272 ng) in saline. At P34, the mice were killed, the eyes enucleated, fixed, processed, embedded, sectioned and stained with hematoxylin and eosin (H&E). Using light microscopy, the thickness of the ONL was measured every 240 μm and these data were plotted vs the distance from the optic nerve head (FIG. 12). The thickness of the ONL in control heterozygous mice is over 50 μm near the center of the eye and decreases as the periphery is approached. The data from the homozygous wild type (not shown) are essentially identical to those of the heterozygous mouse. The ONL of the tubby retina is about half as thick as the control in the saline injected mice but the CNP injected eyes show a significant increase in thickness of the ONL at every point measured. These data demonstrate that intravitreal injection of CNP protects photoreceptor cells in the tubby retina. Based on the ability of CNP to scavenge ROS and our data demonstrating the involvement of ROS in the degeneration of the tubby retina, we concluded that the mechanism by which the CNP are inhibiting the degeneration of the tubby retina involves scavenging ROS.

### Example 11

Intraperitoneal vs. Intracardial Injections of CNP.

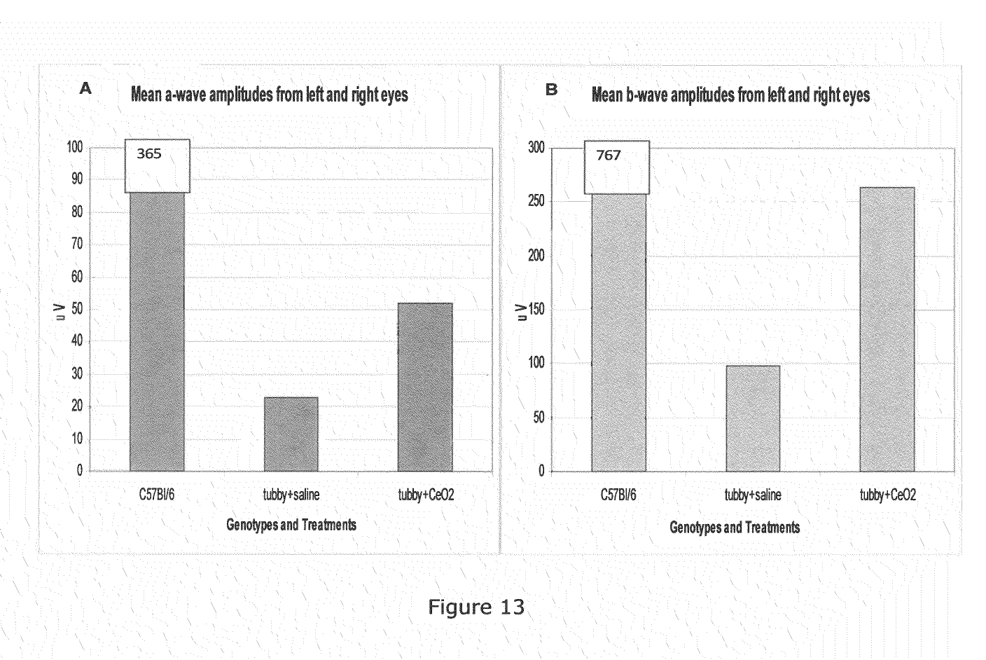

To assess the therapeutic potential of systemic administration of CNP, we used the tubby mouse model to test two systemic modes of administration (1) intraperitoneal and (2) intracardial. To prolong visual function in an inherited retinal degeneration model using a systemic CNP approach, we chose a concentration of CNP that we thought would protect the photoreceptors over extended periods of time. Tubby mice (FIG. 13) were given intraperitoneal injections of 20 uL of a 1 mM suspension of the CNP in saline every day for 27 days, starting at day P7, when their vision was evaluated using electroretinography (ERG). Retina function (both A & B wave) was more than two fold higher in the CNP injected tubby than in the control saline injected tubby mice. These data demonstrate that systemically-administered-CNP do inhibit inherited retinal degeneration and protect retinal function in the tubby mouse.

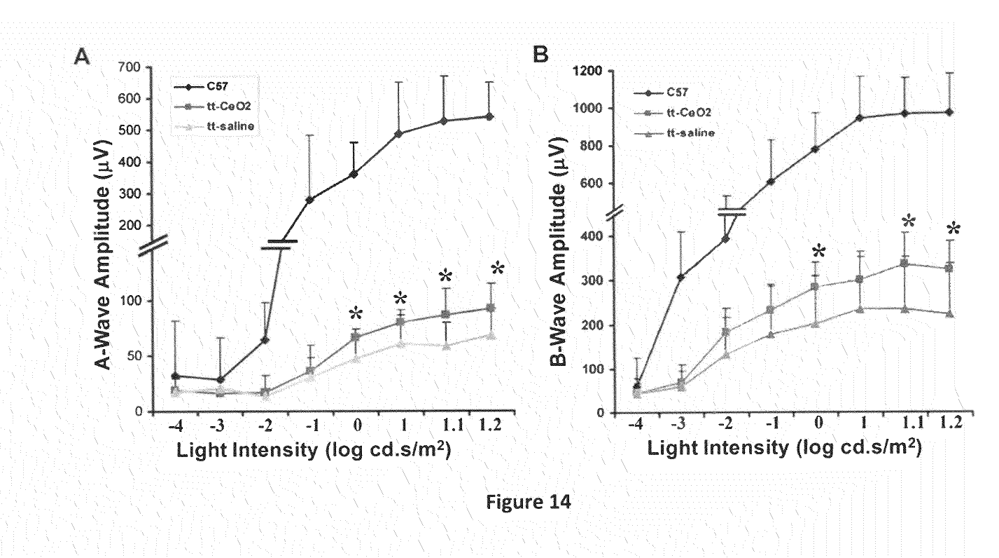

To test whether intracardial injections might be more protective, tubby mice were given three intracardial injections, at days P10, P20 and P30, of 20 ul of either saline or a 1 mM suspension of CNP in saline. Scotopic ERGs were performed on day P34. FIG. 14 shows the results of these functional ERG assays. The maximum response at seven different intensities of light were determined and found to be essentially identical for normal and heterozygous mice. However, the data for the CNP-injected mice indicated an almost two-fold increase in retinal function for both the A-wave and the B-wave measurements.

### Example 12

Protection of Rod and Cone Cells by Nanoceria.

There are 144 different genes in which mutations can lead to retinal degeneration. Rather than developing individual therapies, the strategy contemplated herein is based on a single node, addressing oxidative stress, which is common to these blinding diseases. The tubby mutation in mice causes progressive rod and cone photoreceptor cell death. By four weeks of age (P28), half of the rod and cone cell population has died. Inorganic cerium oxide nanoparticles (nanoceria) have antioxidant activity and can protect mammalian cells from oxidative-stress induced cell death. We determined if nanoceria could prevent rod and cone cell death in this retinal degeneration model.

Nanoceria or saline was delivered to tubby mutants starting at day P7 or P10. Protection from rod and cone cell death was evaluated by electroretinography (ERG) and retinal histology at 4 weeks of age (P28). Oxidative stress level was determined in retinal sections. Levels of pro-apoptotic enzymes and anti-oxidative stress phase II enzymes were also determined.

Oxidative stress markers were significantly reduced in retinas of tubby animals administered with nanoceria. Nanoceria prevented the death of a significant number of rod and cone cells. The rod and cone function in these animals concomitantly showed a significant increase as measured by ERG. Anti-oxidative stress phase II proteins or their activities were up regulated while two pro-apoptotic enzymes were down regulated in the nanoceria injected animals. We have demonstrated that the nanoceria prolong the functional life span of defective rod rod and cone cells in the tubby mouse, thus preserving vision.

As noted, the tubby mutation in mice causes progressive rod and cone cell death. We showed that bright cyclic light (800 lux) accelerated the degeneration of photoreceptor cells in tubby mutants. This suggests that the defective photoreceptor cells may be hypersensitive to oxidative stress. Using a rat light damage model, we demonstrated that inorganic cerium oxide nanoparticles (nanoceria) prevented photoreceptor cell death. Presently we investigated if nanoceria could prevent photoreceptor cell death in this retinal degeneration model.

Mice of genotypes tub/tub, tub/+, and wild type (C57BL/6J) were raised in cyclic light condition. Nanoceria or saline was delivered to tubby mutants starting at P7 or P10 depending on routes of delivery. Three delivery routes were performed: intravitreal (IV), intraperitoneal (IP), and intracardial (IC). Protection from photoreceptor cell death was evaluated by electroretinography and retinal histology at 4 or 5 weeks of age. The degree of oxidative stress was determined in retinal sections using the reactive oxygen species probes dihydroethidium and dichlorofluorescein. We also measured gene expression for proapoptotic enzymes and anti-oxidative stress phase II enzymes in these experimental animals. We showed that intravitreal and intracardial delivery of nanoceria prevented the death of a significant number of rod and cone cells. The rod and cone function in these animals concomitantly showed a significant increase as measured by a- and b-wave amplitudes compared to saline injected animals. We determined that three antioxidative stress phase II proteins or their activities were up regulated while two pro-apoptotic enzymes were down regulated in the nanoceria injected animals. We also demonstrated that oxidative stress markers were present in significantly higher amounts in tubby retinas than in wild type or heterozygous retinas. In support of this, we have demonstrated that the nanoceria prolong the functional life span of defective rod and cone retinal cells such as cells in the tubby mouse, thus preserving vision. It is contemplated in the presently claimed and disclosed inventive concept(s) that nanoceria can be systemically applied to inhibit rod and cone cell degeneration in other mammalian species described herein as well.

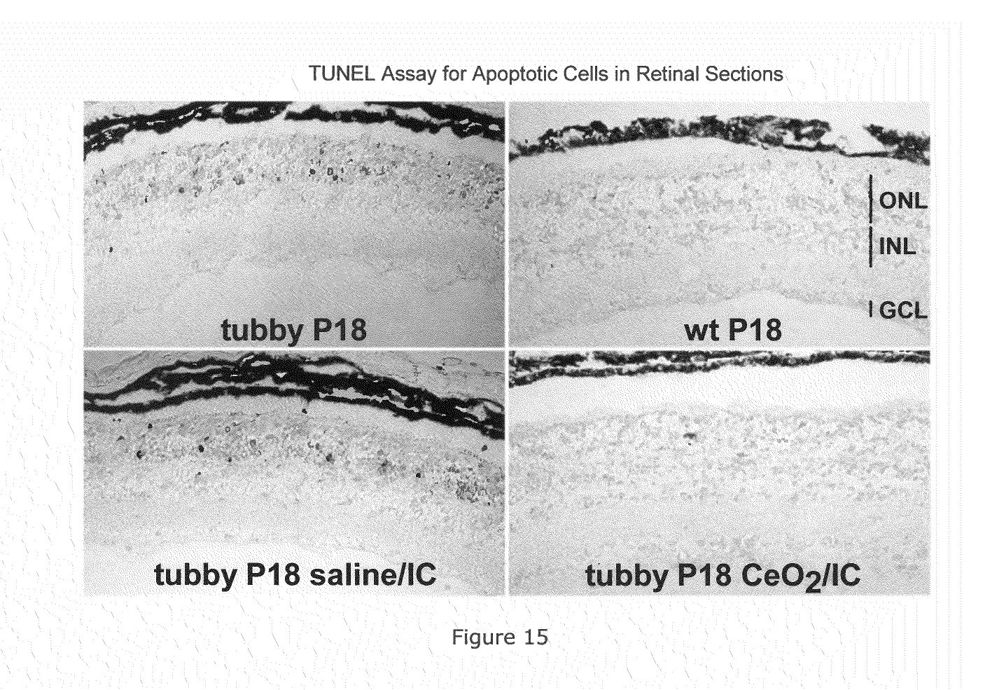

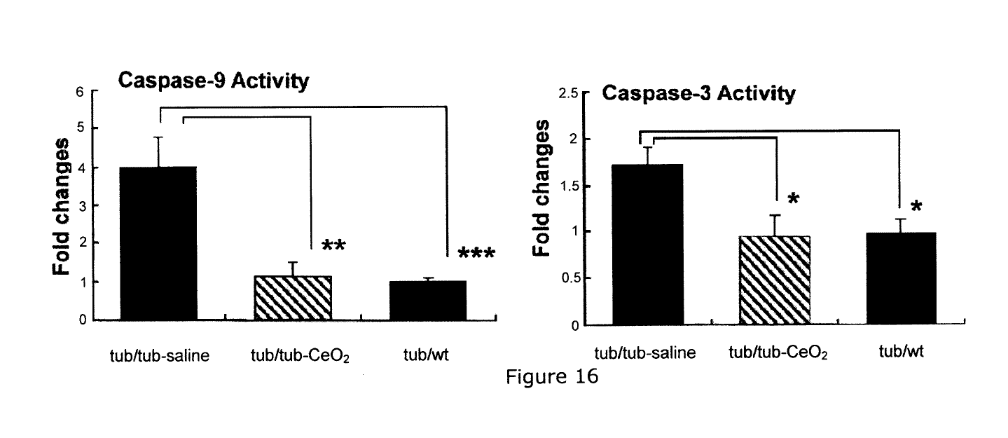

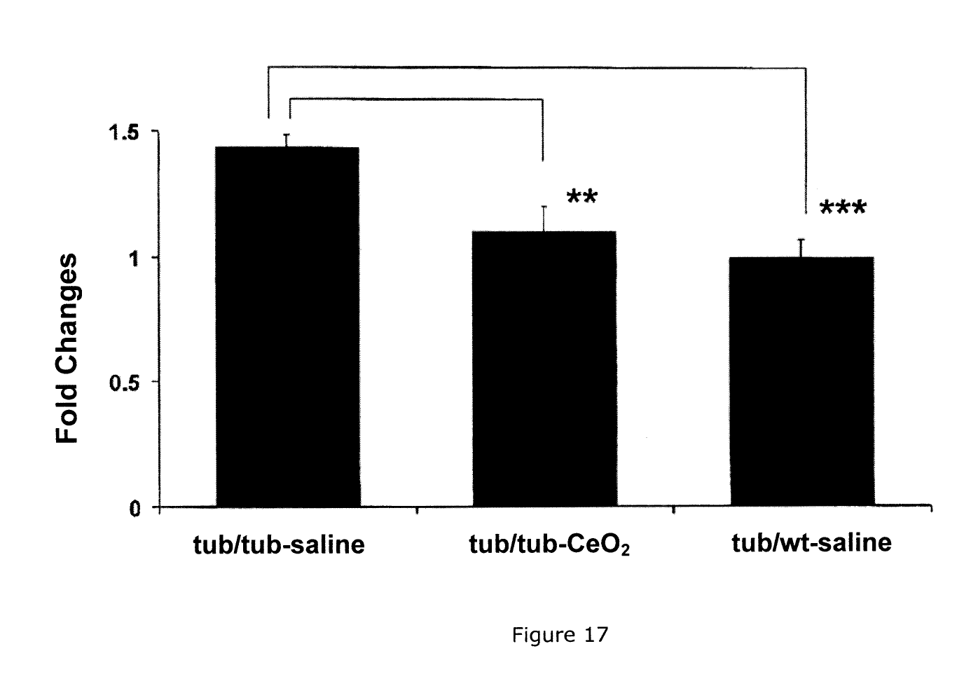

At P18, we observed a significant reduction of apoptotic cells in CNP treated (IC injection) tubby retinas assessed by the TUNEL assay (FIG. 15). At P18, we observed a significant reduction of activated caspases (-9, -8, and -3) in CNP treated (IC injection) tubby retinas (FIGS. 16 and 17). Data in FIG. 16 which represent retinas of tub/tub mice injected with CeO₂ showed significant reduction of Caspase-9 and Caspase-3 activities assayed at P18. There is no significant difference between CeO₂ injected tubby mice and wild type or heterozygotes. Data are expressed and mean±SD (n=4 in each group) (*p<0.05, **p<0.01, ***p<0.001). Data in FIG. 17 were obtained from animals which received 20 μl of saline or 1 mM CeO₂ intracardially. Retinas were collected at P18. Data are expressed as mean SD, n=4 (**p<0.01; ***p<0.001). This is consistent with our findings that more rod and cone cells are present, or fewer rod and cone cell died at P34 of similarly treated tubby animals.

Assays for Reactive Oxygen Species (ROS) Level in Retinal Sections (I)

Dihydroethidium (DHE) Imaging

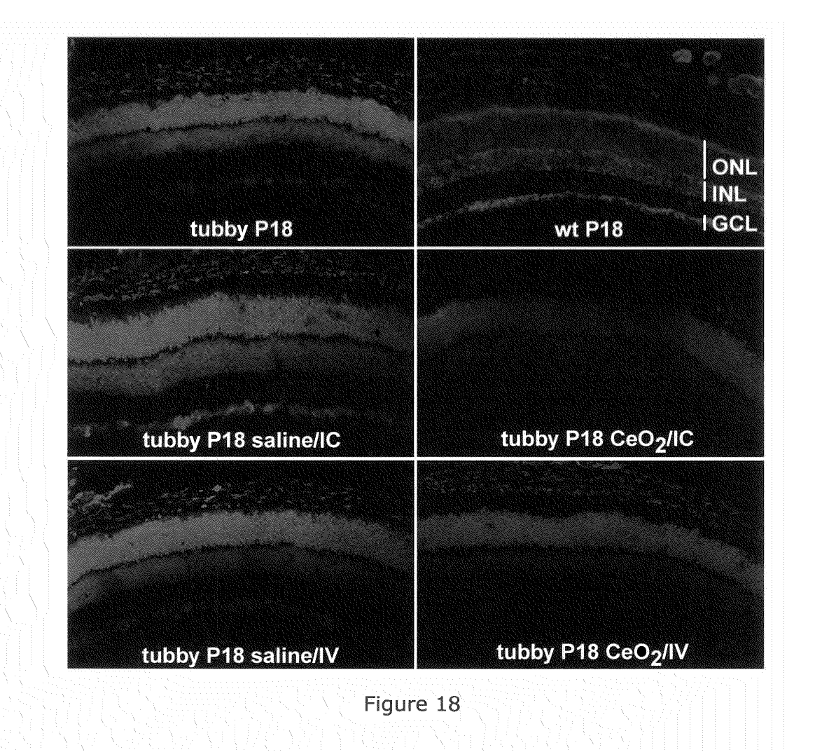

DHE is the reduced form of ethidium bromide. It is oxidized by superoxide to ethidium inside the cell, where it intercalates the DNA and fluoresces at 605 nm (red). ROS levels are much higher in tubby retinas than in wild type at P18 (FIG. 18). ROS levels are significantly reduced in retinas of CNP-treated tubby mice than in saline-treated ones whether treated by IV or by IC injection.

Assays for Reactive Oxygen Species (ROS) Level in Retinal Sections (II):

Dichlorofluorescein (DCF) Imaging

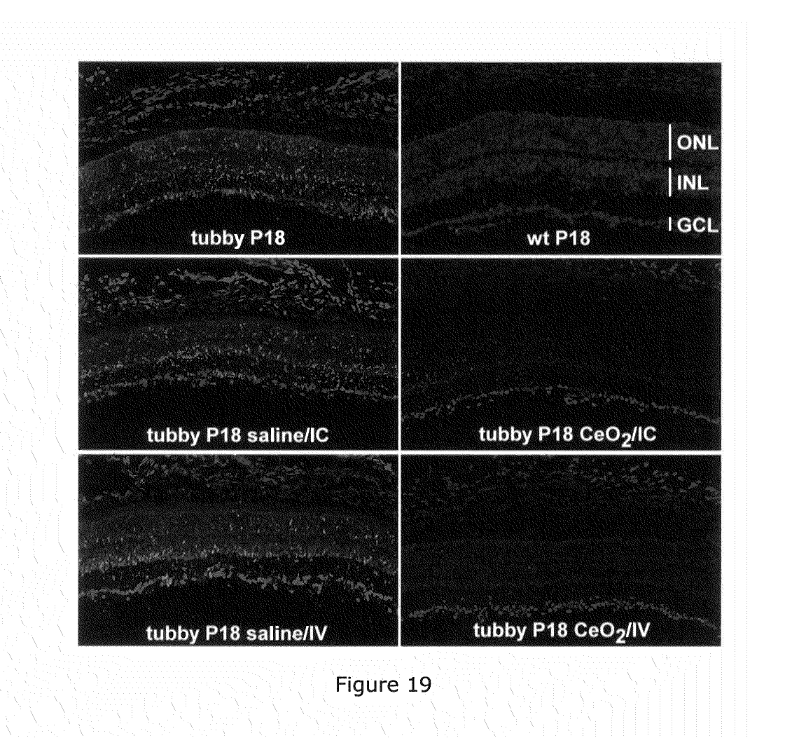

5-(and-6)-chloromethyl-2′,7′-dichlorodihydro-fluorescein diacetate, acetyl ester (CM-H₂DCFDA) is an intracelluar ROS indicator. It is non-fluorescent until the acetate groups are removed by intracellular esterases and its oxidation within the cell. DCF emits a green fluorescent signal. As shown in FIG. 19 nanoceria significantly reduce ROS levels of tubby mice whether treated by IV or by IC administration.

In summary it is shown that both IV and IC delivery methods of CNP (nanoceria) are effective in lowering the ROS levels in the tubby retinas and both IV and IC delivery methods of CNP are effective in reducing the apoptotic death of photoreceptor cells in the tubby retinas. It is therefore demonstrated that either ocular-targeted or systemic administration of CNP is therapeutic for treating retinal degeneration conditions.

Although the presently claimed and disclosed inventive concept(s) and its advantages have been described in detail, it should be understood that various changes, substitutions and alterations can be made herein without departing from the spirit and scope of the presently claimed and disclosed inventive concept(s) as defined by the appended claims. Moreover, the scope of the present application is not intended to be limited to the particular embodiments of the processes, compositions of matter, means, methods and steps described in the specification. As one of ordinary skill in the art will readily appreciate from the disclosure of the presently claimed and disclosed inventive concept(s), processes, compositions of matter, means, methods, or steps, presently existing or later to be developed that perform substantially the same function or achieve substantially the same result as the corresponding embodiments described herein may be utilized according to the presently claimed and disclosed inventive concept(s). Accordingly, the appended claims are intended to include within their scope such processes, compositions of matter, means, methods, or steps.

Each of the references, patents or publications cited herein is expressly incorporated by reference in its entirety.

What is claimed is:

1. A method of inhibiting, reducing or reversing the rate of neovascularization and proliferation of neovascular tissue associated with a pathological condition characterized as having neovascularization in a mammalian subject in need of such treatment, the method comprising:
administering to the mammalian subject a therapeutically effective amount of a composition comprising cerium oxide nanoparticles, and a pharmaceutically acceptable carrier, thereby inhibiting, reducing, or reversing the rate of neovascularization and proliferation of neovascular tissue associated with the pathological condition in the mammalian subject.

2. The method of claim 1 wherein the pathological condition is an ocular condition.

3. The method of claim 2 wherein the ocular condition is characterized by subretinal neovascularization or choroidal neovascularization.

4. The method of claim 2 wherein the ocular condition is at least one of age-related macular degeneration, diabetic retinopathy, retinal angiomatomous proliferation, glaucoma, retinitis pigmentosa, retinal detachment, inherited retinal degeneration, Stargardt's disease, hypertensive retinopathy, and occlusive retinopathy.

5. The method of claim 1 wherein the pathological condition is an inflammatory disease.

6. The method of claim 5 wherein the inflammatory disease is rheumatoid arthritis or osteoarthritis.

7. The method of claim 5 wherein the inflammatory disease is an intestinal disease.

8. The method of claim 1 wherein the pathological condition is a skin disease.

9. A method of inhibiting, reducing or reversing the rate of retinal cell degeneration in a mammalian subject in need of such treatment, the method comprising:
administering to the mammalian subject a therapeutically effective amount of a composition comprising cerium oxide nanoparticles and a pharmaceutically acceptable carrier, thereby inhibiting, reducing, or reversing the rate of retinal cell degeneration in the mammalian subject.

10. The method of claim 9 wherein the mammalian subject has at least one of age-related macular degeneration, diabetic retinopathy, retinal angiomatomous proliferation, macula edema, glaucoma, retinitis pigmentosa, retinal detachment, inherited retinal degeneration, Stargardt's disease, hypertensive retinopathy, and occlusive retinopathy.

11. The method of claim 9 wherein the composition is administered parenterally, intravenously, intramuscularly, subcutaneously, intraorbitally, intracapsularly, intrasynovially, intraperitoneally, intracisternally or by passive or facilitated absorption through the skin by injection into a blood vessel that supplies blood to the eye, or by microinjection into the macula by first penetrating the sclera, by topical application such as to a tissue of the eye such as the cornea or sclera, and/or by implantation such as by controlled release from a depot or implant comprising a pharmaceutically acceptable matrix or pharmaceutically acceptable carrier, which depot or implant is located proximal to the tissue of the eye.

12. The method of claim 9 wherein the composition is administered at concentrations of 1 nM to 1000 μM, or from 1 nM to 100 μM, or from 1 nM to 10 μM, or from 1 nM to 1 μM, or from 1 nM to 100 nM, or from 1 nM to 50 nM, or from 1 nM to 10 nM, or from 10 nM to 10 μM, or from 100 nM to 10 μM.

13. The method of claim 9 wherein the cerium oxide particles range in a size of from approximately 1 nanometer in diameter to approximately 10 nanometers in diameter.

14. The method of claim 9 wherein the retinal cells degeneration which is inhibited, reduced, or reversed is in regard to photoreceptor cells.
